(19) United States
(12) Patent Application Publication
(10) Pub. No.: US 2010/0256671 A1
(43) Pub. Date: Oct. 7, 2010

(54) Tissue sealant for use in noncompressible hemorrhage

(75) Inventor: George Falus, New York, NY (US)

Correspondence Address: BIOMEDICA MANAGEMENT CORPORATION;c/o George D. Falus, PhD, 760 Parkside Avenue, No. 210, Brooklyn, NY, 11226, US

(73) Assignee: BIOMEDICA MANAGEMENT CORPORATION, Brooklyn, NY (US)

(21) Appl. No.: 12/419,734

(22) Filed: Apr. 7, 2009

(51) Int. Cl. A61B 17/03; A61K 47/42; A61K 9/10

(52) U.S. Cl. 606/214; 514/774; 424/484

(57) ABSTRACT

ClotFoam is an hemostatic agent designed for non-compressible hemorrhage. It can be applied outside the operating room through a mixing needle and/or a spray injection method following abdominal, chest, extremities or other intracavitary severe trauma to promote hemostasis, or it can be used in the operating room for laparoscopic procedures or other surgical procedures in which compression is not possible or recommended. Its crosslinking technology generates an adhesive three-dimensional polymeric network or scaffold that carries a fibrin sealant required for hemostasis. When mixed, Clotfoam produces a foam that spreads throughout a body cavity reaching the lacerated tissue to seal tissue and promote the coagulation cascade. The viscoelastic attachment properties of the foam as well as the rapid formation of a fibrin clot that ensure that the sealant remains at the site of application without being washed away by blood or displaced by movement of the target tissue .

### FIELD OF THE INVENTION

The present invention which has been trademarked as “ClotFoam”, is generally related to an adhesive sealant composition and hemostatic agent which may be used to bond or seal tissue in vivo without compression, stitches or staples, and is particularly related to a three component in liquid state which are mixed together as it is applied to tissue and then cured in vivo in order to bond tissue, to seal tissue to prevent or control intracavitary or internal hemorrhage.

### BACKGROUND OF THE INVENTION

Traumatic injury is a frequent cause of morbidity and mortality worldwide. Over 40% of the trauma cases admitted at hospitals in the USA is due to road traffic accidents. Hemorrhage is the primary cause of death on the battlefield in conventional warfare (1). The vast majority of these deaths occur in the field before the injured can be transported to a treatment facility (2). Almost 50% of combat fatalities in Iraq and Afghanistan, and up to 80% of civilian trauma fatalities within the US, are attributed to uncontrolled hemorrhage (3).

The major causes of death in this group are hemorrhage (50%) and neurological trauma (36%), whereas the rest are from devastating multiple injuries. Even when the injured survive long enough to be transported to a medical facility, hemorrhage still remains the leading cause of late death and complications (2). Abdominal injuries pose a formidable problem, especially in young adults (4-8). Being the largest solid organs within the abdomen, the liver and the spleen are the most frequently injured organs (9-10).

Massive bleeding from the liver is currently controlled by Pringle's maneuver or packing of the wound, both of which procedures require surgical intervention and cannot be applied in the battlefield or at the site of accident (11, 12). Splenic trauma ranges can bleed profusely with minimal injury (10-12). Early and effective hemorrhage control can save more lives than any other measure. But since all current haemostatic agents for intracavitary bleeding are designed to be used in the operating room with the cavity wide open (13), not in an emergency at the site of accident or in the battlefield, hemorrhage is often lethal. Also certain types of surgery such as laparoscopic procedures or brain surgery, as well internal bleeding that today require compression could be treated in a less invasive manner.

Current solutions and limitations. Biological glues which can adhere to tissues are known. In general, the synthetic adhesives are used for the tight sealing of vessels or of lungs and for “gluing” the edges of skin incisions. These glues are eliminated, in general after the cicatrisation of the wound, by biodegradation, absorption or by simple detachment in the form of scabs. Various technologies have been developed for the formulation of tissue adhesives. Some of them are of synthetic origin, such as the glues based on cyanoacrylates (2-butyl cyanoacrylate, 2-octyl cyanoacrylate), or on synthetic polymers, and others contain biological materials such as collagen or fibrin which in addition have hemostatic properties and also act by controlling bleeding.

As a result of their hemostatic and adhesive properties, sealants, and particularly fibrin sealants have been extensively used in most surgical specialties for over two decades to reduce blood loss and post-operative bleeding because of the ability to adhere to human tissue as it polymerizes (14, 15, 16). These compounds are used to seal or reinforce wounds that have been sutured or stapled; they can also be used with pressure over an injured area. Fibrin sealants are biological adhesives that mimic the final step of the coagulation cascade. (13) The main components of the sealant are fibrinogen, plasmatic proteins and factor XIII on the one hand and thrombin, and calcium chloride on the other. The components are extracted from human plasma. Mixing fibrinogen and thrombin simulates the last stages of the natural coagulation cascade to form a structured fibrin clot similar to a physiological clot.

There are several commercial products available (Floseal, Gelfoam, Evicel) (16-18). However, these products have significant limitations which have prevented their widespread use in emergency medicine (trauma) and laparoscopic surgery. All existing haemostatic agents for intracavitary bleeding are designed to be used in the operating room and not in an emergency, e.g. at the site of accident or in the battlefield and all require compression. One of the major limitations encountered in the development and/or use of tissue adhesive and sealant compositions for non-compressible hemorrhage is their inability to form a sufficiently strong bond to tissues and to develop a method of application. Therefore, tissue adhesives and sealants have to be employed in combination with compression methods, sutures and/or staples so as to reduce the tissue-bonding strength required for acceptable performance. However, there are many situations where the use of sutures and/or staples is undesirable, inappropriate or impossible. The difficulty of the adhesive matrix to form a strong interface or bond with tissues is most likely due to several factors: The intracavitary free blood or flowing blood does not allow the compounds that promote coagulation to reach the bleeding source, and various proteins in the tissue are not readily amenable to non-covalent and/or covalent interactions with the tissue adhesive or sealant components as applied and/or during and after curing. As a result, for most tissues and adhesive and sealant systems, failures are generally believed to occur at the interface between the cross-linked adhesive matrix and one or more tissue-associated proteins such as collagen, actin and myosin (19,20).

The present alternative approach: Clotfoam. Agents that can achieve hemostasis without compression and/or sutures are required to stop bleeding from severe intracavitary trauma outside the operating room. Non-compressible technologies are also useful in the operating room where compression cannot be applied (e.g. laparoscopic surgery, neurosurgery, etc.). In order to resist the flow of blood, the adhesive matrix must form in a matter of seconds a strong interface and bond with tissues in the midst of flowing blood and remain at the lacerated site to form a clot. The ability of the present agent to adhere to human tissue is related to the internal structure of the scaffold carrying the fibrin sealant that translates into the necessary viscoelastic and adhesive properties as it polymerizes. Rapid formation of the hydrogel, a minimum polymerization time to produce an adhesive gel that contains the necessary components to develop a functional fibrin clot over lacerated bleeding tissue is clinically important. Instant tissue sealant adhesion is desirable to ensure that the sealant functions on contact and remains at the site of application without being washed away by blood or displaced by movement of the target tissue. (21)

In our approach these functions are met through a) the in-situ generation of a three-dimensional polymeric cross-linking chemistries network that is bonded to the tissue, and b) the viscoelastic characteristics of foam, producing a very sticky matrix that attaches to lacerated tissue and wet surfaces; and c) the instant formation of a strong fibrin clot stabilized by Factor XIII. Stickiness and other viscoelastic properties contribute substantially to the ability of the fibrin polymer to stimulate the coagulatory cascade, form a blood clot and achieve hermostasis.

Composition, ClotFoam incorporates fibrin monomer in solution ready to polymerize at change of pH produced by the dialysis method, which is embedded in a hydrogel scaffold. The scaffold is cross-linked in the presence of activated transglutaminase enzyme (calcium dependant and Calcium independent). Both polymers, fibrin and scaffold, when cross-linked in situ, fulfill three objectives or functions: a) allow non-invasive application and dissemination of the agent in the peritoneal or other body cavities; b) adhere and compress lacerated or wound tissue to prevent flow of blood; and c) maintain over the wound the necessary components to produce a fibrin clot and stimulate the coagulatory cascade.

The scaffold uses gelatin as the “structural” protein cross-linked with biological polymers to achieve a specific viscoelastic profile that is ideal for carrying the fibrin monomer and for neutralizing its pH in order to polymerize it. (21, 22). When polymerized by the mixing of the parts, fibrin provides a critical provisional matrix at sites of injury (23).

The non-invasive application and dissemination is based on the production of foam upon mixing the components, which once injected spreads throughout the cavity reaching the lacerated tissue to stimulate the blood coagulation cascade. Other important differences with existing gels are that the proposed adhesive uses as a cross-linked structural protein, Teleostean fish gel gelatin Type A, Bovine serum albumin (BSA, protein), Carbomer 934 (polyacrylic acid crosslinked with perallyl sucrose), a calcium-independent crosslinking catalyst (MTGa) and alternative materials such as polysaccharides and polyvinylpyrolidone, Carrageenan (sulfonated polysaccharide) sucrose, MgCl₂, with or without Alginic acid, Carboxymethyl cellulose, providing better and “intelligent” cross-linking chemistries that modify the liquid-gel state and viscosity as needed.

The ability of the matrix to achieve hemostasis depends not only the formation of fibrin itself, but also on interactions between specific-binding sites on fibrin, pro-enzymes, clotting factors, enzyme inhibitors, cell receptors and, equally importantly, the dynamics of distribution and viscoelastic attachment properties of the foam (24,25). The activity of these factors can be enhanced or improved to produce a strong clot able to stop the parenchimal bleeding in the spleen, liver and other solid organs in the abdominal cavity, cranial cavity, and soft tissue. Each part is formulated to maximize the activity of fibrin clot component.

The instant gelation of the scaffold and its ability to rapidly produce a fibrin clot is essential to ensure that the sealant remains at the site of application without being washed away by blood or displaced by movement of the target tissue (19). Under coagulant conditions, additional factor XIII, as well as Ca (2+), Mg++ and Zn++, contribute to this process by stabilizing the fibrin clot through covalent bonds. (24)

Key Attributes. Polymerization/Adhesion. The gel foam is formed as a result of the covalent cross-linking of the gelatin chains, serum albumin and carbomer 934 in the presence of sucrose, metallic ions, and calcium independent transglutaminase enzyme. [24]. The gel carries and supports the polymerization of fibrin monomer in solution, which is stabilized by Ca++ activated Factor XIII into a fibrin clot within 1 minute of application. The clot is mechanically stable, well integrated into the scaffold, [25] and more resistant to lysis by plasmin compared with an uncross-linked clot [26] or other fibrin sealants. Components of the scaffold together with Factor XIII facilitate the transglutaminase-mediated oligomerization of the aC-domains of fibrin promoting integrin clustering and thereby increasing cell adhesion and spreading, which stimulates fibrin to bind avb3-, avb5-and a5b1-integrins on EC (27). The oligomerization also promotes integrin-dependent cell signaling via focal adhesion kinase (FAK) and extracellular signal-regulated kinase (ERK), which results in an increased cell adhesion and cell migration, over time, powered by the effects of fish gelatin on fibroblast differentiation [28]. The presence of additional Ca+ and Zinc enhance the progression between the inflammatory response and the coagulation cascade (first stage).

Gelling time under 10 seconds of the scaffold, gel strength over (g′) 2000 dyn/cm2, ability to maintain contact adhesion in wet surfaces and rapid polymerization of the fibrin monomer and stabilization (formation of covalent bonds in the presence of activated Factor XIII) to achieve a functional fibrin clot is clinically important. High tensile strength and adhesive strength are mechanical properties characterizing the gelatin-fibrin polymeric network produced by the agent, which is necessary for successful sealing (29).

The adhesion characteristics to vital human tissue and the kinetics of polymerization of the proposed agent have been tested in vitro and in vivo. The data obtained provides ample evidence of the ability of Clot Foam to stop bleeding and achieve hemostasis with no compression in induced intraperitoneal non-compressible secondary to grade IV traumatic liver damage in rodents and swine models. Depending on the protein concentrations, dilution and catalysts, the gel process begins within 6 seconds of mixing the liquid solutions, reaching gel strength of 2900 dyn/cm2 in 20 seconds. Gel state remains stable between 10 to 20 minutes depending on the concentration of surfactants (tween 80) and pH to finally return of the liquid state. This final state transition facilitates absorption and elimination of agent from the cavity. Studies of tensile static and dynamic loading of the adhesive hydrogels in bulk form demonstrated that the Young's modulus ranged from 45 to 120 kPa and that these bulk properties were higher than to those reported for hydrogels obtained from fibrin-based sealants (28). Even after being washed away, strands of ClotFoam remained attached to both of the opposing lacerated tissues.

Protein gelation. Another important component that ensures the binding of the three-dimensional polymeric network to the tissue surrounding the wound is the structural protein. ClotFoam contains Teleostean Gelatin type A in liquid phase. The raw material for the production of this gelatin is the skin from deep water fish such as cod, haddock and Pollock. It is a protein derived by a mild partial hydrolysis at relatively low temperature from collagen.

The uniqueness of fish gelatin lies in the amino acid content of the gelatin. Although all gelatins are composed of the same 20 amino acids, there can be a variation in the amount of imino acids, proline and hydroxyproline. With lower amounts of these imino acids, there is less hydrogen bonding of gelatin in water solutions, and hence a reduction in the gelling temperature. Gelatin from cod skin gels at 10° C., whereas gelatin from carp skin would be more similar to animal gelatin, which gels above room temperature. Two of its most useful properties are gel strength and viscosity (30).

Biomacromolecules like gelatin have emerged as highly versatile biomimetic coatings for applications in tissue engineering (31). The steady-state adhesion energy of 3T3 fibroblasts on gelatin film is three times higher than that on chitosan film. The better attachment of 3T3 fibroblast to gelatin is postulated to be caused by the presence of adhesive domains on gelatin. Thus, bioabsorbable gelatin and polysaccharides can be used to prepare a safer and stronger hemostatic gel (32).

The sealing effect of rapidly curable #-D network of gelatin-Carbomer -BSA hydrogel glue on lacerated tissue has been studied in our laboratory. Upon mixing of the polymer components in aqueous solution, Schiff base is formed between the amino groups in the modified gelatin and the aldehyde groups in the modified polysaccharides, which results in intermolecular cross-linking and gel formation. The gel formation can take place within 5 seconds, and its bonding strength to it is about 225 gm cm(−2) when 20 wt % of an amino-gelatin (55% amino) and 10 wt % of aldehyde-HES (>84% dialdehyde) aqueous solutions were mixed. Hydrogel glue resulted in superior sealing effect.

Gelatin is widely used in medical applications. Together with water, it forms a semi-solid colloidal gel. It has been already used in several life supporting applications such as plasma expanders and blood substitutes. (31) Gelatin has been suggested as a low effect molecule in the hemostatic variables when utilized as a volume-blood substitute intravenously in hemorrhagic shock (33). This molecule has been related as an excellent natural attachment site for cells as well as a material with a high degree of biocompatibility and readily available to incorporate agents to it that are related to the wound healing process and coagulation.

Viscoelastic properties. Viscosity and elastic moduli at the gel point vary at differing gelatin, carageneen, carbomer and active concentrations. These parameters provide a measure of the flow properties and gel strength at a single time, the gel-point, and provide an indication of optimal distribution of the foam in the cavity and ability to spread throughout the cavity stick to the lacerated tissue and trigger the blood coagulation cascade. The optimal concentration of components as described below allow the adhesive to flow into and mechanically “interlock” or stick to the tissue in order to seal the wound. While a lower viscosity adhesive may lack sufficient cohesive strength to be retained where it is applied and it may be washed away, a higher viscosity formulation may not produce sufficient foam to cover the cavity or be fluid enough to reach the tissue. This problem can be particularly important if the adhesive must be applied to wet tissue. In addition, stronger gels or gels that polymerize faster have greater cohesive strength but might not effectively penetrate and interlock with tissue. Thus, the adhesive's flow properties and gel strength are practically important and the values are defined by the intracavitary non-compressible situation in which an organ or tissue is perforated. (19-21).

The sticky, gummy consistency of the agent maintains the foam in situ over lacerated tissue despite the flow of blood, while PVP and other large molecules enhance the physical adhesion of the foam to wet tissue. The foam property allows for more extensive attachment than would be achievable from a homogeneous liquid form and also provides a scaffold for the growing fibrin network that binds sundered tissue and forms a barrier to blood flow. The incorporation of commercially available bacterial- or plant-derived carbohydrate-based gel components could further enhance the property of ClotFoam. Lo-acyl Gellan gum with calcium ion, alginic acid (pKa=5), and carrageenan with locust bean gum and potassium ions, known to form robust hydrogels, may bed added to better achieve hemostasis in pooled blood.

Fibrin monomer polymerization: An experimental method for producing fibrin monomer was first described and published by Belitser et al (1968, BBA) (34). This method limits the production of monomer to a few milligrams per day. Although U.S. Pat. No. 5,750,657 to Edwardson et al. describes a method of preparing a fibrin sealant utilizing a fibrin monomer composition, the ClotFoam sealant composition, neutralization of the fibrin monomer to produce a polymer, and use of fibrin monomer produced by the dialysis method, is entirely novel. The patent application for this novel method of producing fibrin monomer in industrial quantities was filed simultaneously with the present application. The preparation, properties, polymerization, equilibria in the fibrinogen-fibrin conversion, solubility, activation and cross-linking of fibrin monomer has been studied by several authors since 1968 (35-43).

The composition of parts and method of production of the fibrin monomer are critical to the performance of a non-compressible technology. The power to stick to the lacerated tissue in a pool of blood depends on the cellular and matrix interactions. The characteristics of the fibrin itself, such as the thickness of the fibers, the number of branch points, the porosity, and the permeability and other polymerization characteristics define the interactions between specific-binding sites on fibrin, pro-enzymes, clotting factors, enzyme inhibitors, and cell receptors [24]. For example, more coarse matrices show a faster fibrinolysis and the pH of the fibrin matrix determines the in-growth of tubular structures. Opaque matrices at pH 7.0 consist of thick fibers and tube formation proceeds at a faster rate than in transparent matrices at pH 7.8 that consists of thinner fibers [20]. Several conditions may affect the fibrin structure, such as the clotting rate (can be modulated by concentration of thrombin and salt content), but also by the presence of metallic ions, proteins and enzymes, the rate of polymerization (determined by FXIII concentration and FXIII activation rate), and the rate of lateral polymerization (affected by fibrinopeptide B release and cross-linking sites on alpha and gamma chains). Chloride and Zn ions have been identified as modulators of fibrin polymerization, because these ions control fiber size by inhibiting the growth of thicker, stiffer, and straighter fibers. The concentration of thrombin to produce a fibrin monomer and thus the release-rate of FPA can also have an important impact on the polymerization process. High concentrations (up to 1 U mL)1) induce the formation of thin fibers, whereas low concentrations (0.001 U mL)1) result in thick fibers [20]. Clot foam part C (fibrin monomer) mixed with part A and B carry components in concentrations, dilutions and pH that will produce an optimal fibrin structure at an accelerated rate.

pH—Studies conducted by other investigators (34) and our own investigations, demonstrated that a pH and ionic strength dependency on polymerization and crosslinking of the scaffold and fibrin monomer and therefore clot formation existed. pH determines the viscosity of solution comprising the scaffold and the ability of the solution to neutralize the acid pH of monomer solution, thereby producing a polymer. That will be stabilized by factor XIII. Clot foam parts A, B, and C are formulated to maintain optimal pH to favor the incorporation, preservation and activity of fibrin sealant components; fibrin monomer, factor XIII, Activa.

Role of the Foam The complementary process that allow the compounds to reach the bleeding source or remain at the lacerated site to form a clot is triggered by an organic non-toxic non-exotermic reaction producing a sticky foam that spread throughout cavity in the same way that sealing foams are use to repair tires. Sodium monobasic phosphate (NaH₂PO₄, is used to buffer pH of Solution B to promote foaming when mixed with part A by acid-base neutralization of the NaHCO₃ and alginic acid, or Carbomer 934. The volume expansion produced by the foam triggering component is from 300% to 400% of the original volume within 10 seconds of mixing solutions. These time frames, strengths and volumes are convenient in the sense that they allow ClotFoam solutions to generate a foam that is distributed throughout the cavity in the form of a strong gel that adheres (sticks) to the lacerated tissue. Our studies have determined the concentration of components necessary to adjust the gel time and gel phase duration (25).

Role of divalent metal ions. The ClotFoam kit in its present form contains Calcium, Zinc and Magnesium ions. It has been established that these ions can markedly increase the rates of fibrin polymerization, and the length and strength of fibrin filaments. The presence of additional Ca+ and Zinc enhance the progression between the inflammatory response and the coagulation cascade. Zn+ modulates fibrin assembly and plays a role in the activation of thrombin-activatable fibrinolysis inhibitor. Ca++ activates Factor XIII

### SUMMARY OF THE INVENTION

The present invention lies within the domain of biological adhesives and tissue sealants, which are biodegradable and nontoxic, intended for therapeutic use, for example, as an intracavitary hemostatic agent for non-compressible hemorrhage.

In one aspect, the present invention relates to biocompatible fluid adhesive protein foam as well as to biocompatible fluid/foam adhesive, which is bio-reabsorbable and nontoxic, for surgical or therapeutic use. It also relates to such foam containing bioactive substances which can be released in a given site.

In another aspect, the invention relates to a process for producing such an adhesive foam and to a kit for the preparation thereof. In yet another aspect, the invention relates to the use of the adhesive foam outside the operating room, in surgery and/or for therapeutic purposes, in particular for protecting wounds and attaching biological tissues or to an implanted biomaterial.

Extensive in vivo studies show that ClotFoam is an excellent hemostatic agent candidate for emergency situations and combat trauma as well for non-invasive surgical procedures. If needed It can be applied by paramedics, it is durable, possess minimal risk, require little training to use, is effective against severe bleeding that would otherwise lead to exsanguination, and capable of sustaining hemostasis for at least several hours to permit safe evacuation to definitive care centers. The application through a “mixing needle” specially designed for non-invasive use is safe, and can be performed in the battlefield or in a medical facility. Thus, a liquid “intelligent” hemostatic” agents that transits from liquid state to gel and back to liquid as needed, is a novel concept since there is no other compound that can be delivered through a needle in a minimally invasive procedure, reaching the injured tissue within the abdominal or other cavity through pooled or flowing blood. ClotFoam also meets the requirements for battlefield use because it is low-weight, low-volume, and low-cube; and requires minimal mixing of components; can be stored, transported and used at environmental temperature by medical personnel at the training level of combat medics

### BRIEF DESCRIPTION OF THE DRAWINGS

FIG. 1 ClotFoam 3-parts applicator and mixing needle

FIG. 2. Mechanism of action

FIG. 3. Denaturation of Gelatin

FIG. 4. View of application

FIG. 5. Introduction procedure for abdominal intracavitary bleeding

FIG. 6. Graph of intratissular adherence

FIG. 7. Graph of Clot strength

FIG. 8 Changes in Foam volume expansion.

FIG. 9 Rheometry of replacement of organic acids (Alginic acid Vs Carbomer)

FIG. 10 Rheometry of formulation with crosslinking agents (Activa)

FIG. 11 Rheological studies on surfactants

FIG. 12-13. Polymerization, cross-linking and stabilization of fibrin in the presence of ACTIVA and Activa+Factor XIII .

FIG. 14 Polymerization and crosslinking of fibrin within the scaffold (part A+part B+part C)

FIG. 15 Agar plates incubated with sterilized and non-sterilized ClotFoam.

FIG. 16 Microphotograph of incubation of Human fibroblasts with ClotFaom (biocompatibility)

FIG. 17 Microphotograph of incubation of Human epithelial cell line A549 with ClotFoam (biocompatibility).

FIG. 18. SDS-PAGE of fibrin monomer (Medved) shelf life

FIG. 19 SDS-PAGE of fibrin monomer (Biomedica) shelf life

FIG. 20 Clotability graph. Shelf life.

FIG. 21. Rheometry 1 to 25 days of ClotFoam gelation. Shelf life

FIG. 22 Images of 1-25 day gelation of Clotfoam.

FIG. 23 Livers were removed for observation of the lesions and clot forming effect in control. Treated livers show the formation of very strong clots in the injured areas

FIG. 24 Blood loss measurement of treated and untreated animals

FIG. 25. Aortic surgical model and Clotfoam application

FIG. 26 Cava vein surgical model and Clotfoam application

FIG. 27 Close cavity model by ultrasound

FIG. 28. Describes a high-speed liver injury drill and sharp rotary drill bit used to produce a traumatic injury grade IV in the liver.

FIG. 29 Grade IV liver injuries through an open midline laparotomy and open cavity ClotFoam application (pig model)

FIG. 30. Grade IV liver injuries produced by a drill through a laparoscopic procedure and closed cavity ClotFoam application (pig model)

FIG. 31. Trend of mean arterial pressures (MAPs) in treatred animals

FIG. 32. Trend of mean arterial pressures (MAPs) in non-treatred animals

FIG. 33. Liver sections (histology) stained by various procedures

FIG. 34. Diagram of component interaction

### DETAILED DESCRIPTION

We have developed an intracavitary hemostatic agent in liquid form, CLOTFOAM, and a method of application for the use in non-compressible hemorrhage outside the operating room or in non-invasive surgical procedures.

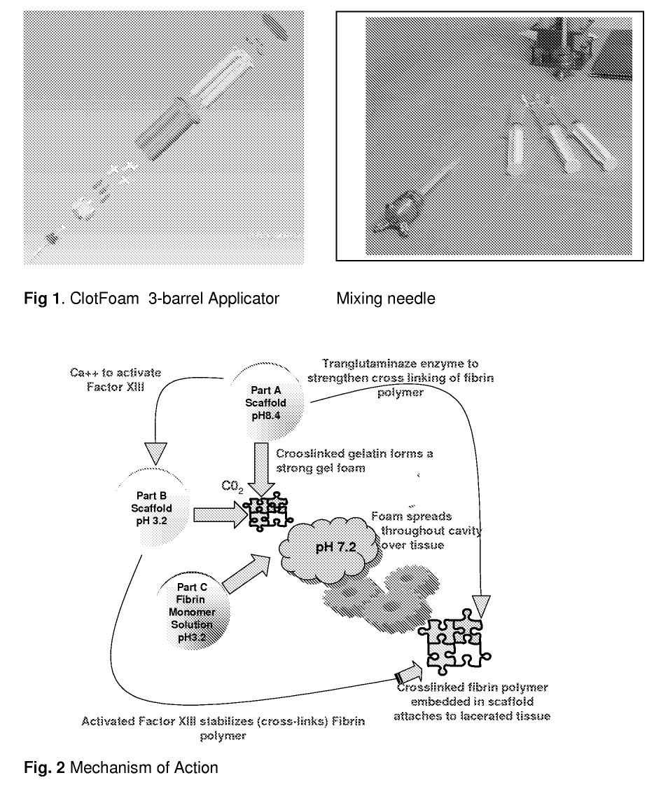

ClotFoam is a novel gelatin based fibrin sealant designed to promote hemostasis in cases severe bleeding and to stop hemorrhage without compression resulting from organ resection, trauma and/or intracavitary wounds grade IV/V or, solid organ wounds, soft tissue and brain that otherwise may lead to clinical complications or exanguinations. CLOTFOAM is intended to be used specifically but not exclusively as an hemostatic agent for emergency situations and combat trauma, and for minimally invasive surgical procedures such as laparoscopic surgery. This sealant agent promotes coagulation and provides hemostasis as well as adhesiveness between surfaces of damaged tissue. ClotFoam is a novel concept since there is no other compound that can be 1) delivered through a mixing needle, 2) sustain hemostatsis for several hours, 3) used when sutures and/or staples are undesirable, inappropriate or impossible, and stop the bleeding and promote early adherence of damaged tissue, and 4) reach the injured tissue within the abdominal cavity through pooled or flowing blood. CLOTFOAM can be used: a) immediately after trauma in the battlefield or at the site of the accident; b) can be applied by paramedics; c) can maintain its viscosity at a wide range of temperatures. This agent meets the requirements for battlefield use because it is low-weight, low-volume, and low-cube; and requires minimal mixing of components; can be stored, transported and used at environmental temperature by medical personnel at the training level of combat medics. CLOTFOAM is a kit (FIG. 1) comprising of three parts solubilized in aqueous medium than when mixed form and adhesive compound which is polymerized/crosslinked, by a polymerization/crosslinking reaction with gelatin(scaffold), and by the additional polymerization of fibrinogen activated by thrombin and stabilized by factor XIII forming a fibrin structure within the gel.

The process that allows the agent to reach the bleeding source or remain at the lacerated site to form a clot is initiated by the mixture of liquid components A, B, and C that when mixed form a gel that is injected into cavity. Once the solutions are mixed, they form a non-toxic low-exotermic reaction that produces a sticky foam which spreads through out the cavity in the same way that sealing foams are use to repair tires. The sticky, gummy consistency of the agent maintains the foam in situ over lacerated tissue despite the flow of blood rapidly forming an adhesive matrix over damaged tissue that 1) seals the wound with a solid cap, 2) triggers the coagulatory cascade to form a blood clot along the wound, and 3) binds together the lacerated tissue.

Clot Foam has been developed in several formulations which vary in foaming volume, viscoelastic properties and clotting strength as needed.

The hemostatic properties of CloFoam formulations are based on the physical and coagulation properties of a scaffold (PART A +PART B) mixed with fibrin monomer in acid solution (PART C).The scaffold consisting of a mixture of Teleostan Gelatin type A in liquid phase, Bovine serum albumin (BSA, protein), Carbomer 934 (polyacrylic acid crosslinked with perallyl sucrose, or Alginic acid (polysaccharide), Carrageenan type 2, Carboxymethyl cellulose albumin, Polyvinylpyrrolidone, and Sucrose mixed with a water-soluble foam inducer of sodium bicarbonate and dihydrogen phosphate; foaming enhancers such as sodium lauryl sulfate, sodium lauroyl sarcosinate, taurate salts and betaine surfactants; and the addition of thrombin, calcium independent transglutaminase enzines such as Activa, Ca, Mg and Zn ions, and fibrin sealant components such as fibrinogen, thrombin, fibrobronectin and Factor XIII.

The mechanism of action illustrated in FIG. 2 shows how Part A mixed with part B cross-links gelatin and BSA to form a strong foaming gel that expands the original volume by 400%. While the gel is being formed, Part A delivers the calcium ions necessary to activate Factor XIII. The fibrin monomer at acidic pH contained in part C is neutralized by Parts A and B. Activated Factor XIII contained in B crosslink the fibrin polymer created following pH neutralization. The ability to remain at the site despite the flow of blood, form a matrix exclusively over lacerated tissue and seal the wound is achieved through a) the surface adhesion and viscoelastic characteristics of a foam components, producing a very sticky matrix carrying the fibrin polymer that attaches to lacerated tissue and wet surfaces and contributes substantially to the ability of the agent to achieve hermostasis, and b) the in situ generation of a three-dimensional polymeric cross-linking chemistry network in the presence of activated Factor XIII, that forms a fibrin clot which is bonded to the tissue.

The components that contribute the formation of the foam are: sodium bicarbonate, dihydrogen phosphate, Alginic acid/or Carbomer 934 and foaming enhancers such as sodium lauryl sulfate, sodium lauroyl sarcosinate, taurate salts and betaine surfactants

The Following components provide the physical viscoelastic properties: Teleostan Gelatin type A in liquid phase, Bovine serum albumin (BSA, protein), Carrageenan type, Polyvinylpyrrolidone, Sucrose and Fibrinogen. The ClotFoam components form a 3-D polymer crosslinked network with high affinity to collagen. The complementary presence of additional Ca, Mg and Zinc ions enhance the progression between the inflammatory response and the coagulation cascade. Zn+ modulates fibrin assembly and plays a role in the activation of thrombin-activatable fibrinolysis inhibitor.

Example of ClotFoam Formulation #10.

ClotFoam is produced by the combination of 3 solutions: A,B and C

Solution A:

Step 1 . Preparation of Neutral A:
micromolar range of ZnCl
Millimolar range MgSO4
Teleostan cold water fish gel
sucrose
polyvinylpyrrolidone
H2O,
all contents are stirred to homogeneity and then the solution is
neutralized to pH 7.1 with NaOH

Step 2. preparation of Final solution A
Neutral A″ (above)
CaCl2
Carrageenan, type 2
NaHCO3
bovine serum albumin
(optional)-. fibronectin

All components are stirred, resulting in a suspension, which is then homogenized with three strokes of a ounce homogenizer.
(optional)-0.02 mg fibronectin

All components are stirred, resulting in a suspension, which is then homogenized with three strokes of a ounce homogenizer.

Step 3) preparation of Solution B
NaH₂PO₄
Tris-Base
Activa
Carbomer 934
FXIII

Step 4) preparation of Solution C

90-120 mg/ml Fibrin monomer solution in 0.125% ice cold AcOH (pH 3.4) are prepared by dyalisis method.

Upon mixing of the components A and B in aqueous solution, Schiff base is formed between the amino groups in the modified (fish) gelatin used in the CLOTFOAM composition, and the aldehyde groups in the modified polysaccharides, which results in intermolecular cross-linking and gel formation. The gel formation can take place within 5 seconds, and its bonding strength to it is about 2000 dyn/cm2 when 20 wt % of an amino-gelatin (55% amino) and 10 wt % of aldehyde-HES (>84% dialdehyde) aqueous solutions are mixed, resulting in superior sealing effect.

ClotFoam gelatin is produced from fish skin, and it is usually referred to as type ‘A’ gelatin. The raw material for the production of this gelatin is the skin from deep water fish such as cod, haddock and Pollock. It is a protein derived by a mild partial hydrolysis at relatively low temperature from collagen. Two of its most useful properties are gel strength and viscosity, on which it is mainly assessed.

TABLE 1
Specifications for ClotFoam gelatins:

| | |
|---|---|
| pH | 3.8-5.5 |
| Isoelectric Point | 7.0-9.0 |
| Gel strength (bloom | 50-300 |
| Viscosity (mps) | 15-75 |
| Ash (%) | 0.3-2.0 |

The gelatin type used in the composition of CLOTFOAM is one of the most pure and perfect protein available, it is absolutely harmless, it is active and readily and rapidly accepted by the body. It is widely used in medical applications. Together with water, it forms a semi-solid colloidal gel. It has been already used in several life supporting applications such as plasma expanders and blood substitutes. (29)

The uniqueness of fish gelatin lies in the amino acid content of the gelatin. Although all gelatins are composed of the same 20 amino acids, there can be a variation in the amount of imino acids, proline and hydroxyproline. With lower amounts of these imino acids, there is less hydrogen bonding of gelatin in water solutions, and hence a reduction in the gelling temperature. Gelatin from cod skin gels at 10° C., whereas gelatin from carp skin would be more similar to animal gelatin, which gels above room temperature. Fish gelatin can be reacted with anhydrides under alkaline conditions, reducing or eliminating the effect of aldehydes as a hardening agent on the gelatin

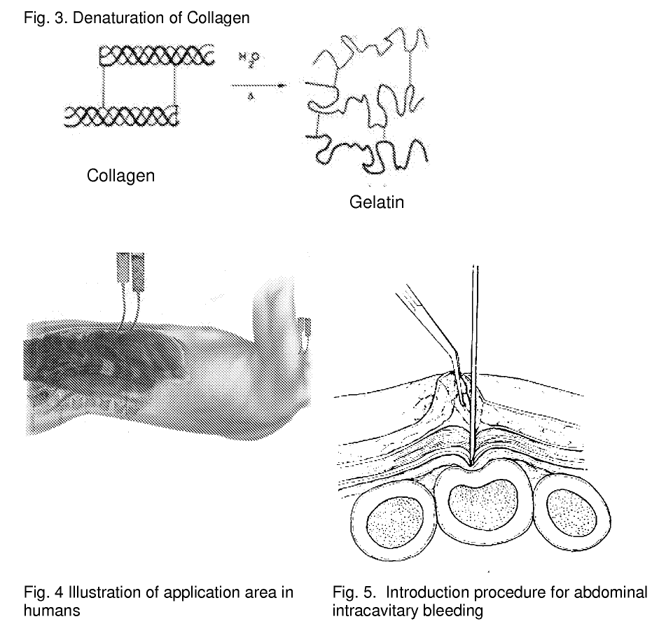

Boiling hydrolyzes the collagen, and converts it into gelatin. (FIG. 3) Two processes are used: an acid process gives type A gelatin and an alkaline process gives type B gelatin. Their properties are similar, but type A can negatively interact with other anionic polymers, and this is the chemical feature that gives CloFoam its adhesiveness properties (adhesion to lacerated tissue).

There is also an important relationship between the temperature at which the fish metabolizes and the properties of the skin and the resultant extracted gelatin. Gelatin derived from the skin of deep cold water fish has lower amounts of proline and hydroxyproline, and as a result, water solutions will not gel at room temperature, but will remain liquid to 8 to 10° C. There are similarities between the gelatins, but most animal gelatin gels at 32° C., whereas the fish gelatin remains liquid down to 8° C. This property is very useful to produce a product with long shelf life and the capability to be stored at room temperature in its liquid physical state. Also, It is important to be able to keep the solubility of the product in a wide range of temperatures in order to be readily to be activated in any environment of battlefield, whether cold or warm weather.

Delivery Methods:

ClotFoam is delivered into the cavity by the CO₂ propelled applicator through a mixing needle. (FIG. 4) The following procedure, which is designed for use in cases of severe intracavitary trauma (liver injury grade III/IV) can be adapted for use in laparoscopic, urinary track, gynecological or brain surgery as well as for other minimally invasive procedures.

The mixing needle introduction procedure for abdominal intracavitary bleeding (FIG. 5):

1. Preparation of the Abdomen: The sub-umbilical area on the anterior abdominal wall should be prepared with iodine wipes and a sterile field.

2. Insertion of the mixing Needle: The sub-umbilical area at the anterior abdominal wall is the thinnest at this level and all fascial layers are fused into single fascial planes. Thus, the operator should always attempt to insert the needle at this site in the virgin abdomen.

3.—Elevating the Anterior Abdominal Wall The anterior abdominal wall needs to be elevated in order to distance it from the intra-abdominal contents. This is done by grabbing the abdominal wall directly under the umbilicus with one hand

4.—The Incision A 1 mm incision is made with a #11 Scalpel below the of the umbilicus.

5.—Insertion of the needle: The needle is then slowly inserted into the incision. It is angled toward the pelvis and advanced. The operator should feel or sense the needle passing through two distinct planes. The needle is advanced and withdrawn several times. If this is done easily and without obstruction, the tip is in proper position.

6.—The Saline Test. Ten cc of normal saline is injected. This should be done easily. The abdominal pull is then released. The needle is then fixed to the abdominal cavity with surgical tape and then connected to the C02 gas-propelled delivery gun designed to mix and activate the solutions that injects a total of 150 to 210 cc of ClotFoam at average pressure of 100 PSI. Once the foam is totally squeezed into the abdomen the needle is removed and gauze is placed and fixed with tape.

### EXAMPLES

The adhesion characteristics to vital human tissue and the kinetics of polymerization of the gel have been tested in laboratory studies showing that the CLOTFOAM sealant polymerizes faster than other sealants. Rheological measurements indicate that CLOTFOAM catalyzes the conversion of gelatin solutions into hydrogels, and gel times are on the order of 6 seconds. G′ reaches 2000 dyn/cm2 in less than 10 seconds. Studies of tensile static and dynamic loading of the adhesive hydrogels in bulk form demonstrated that the Young's modulus sometimes referred to as “Modulus of Elasticity” ranged from 175 to 240 kPa and that these bulk properties were stronger to those reported for hydrogels obtained from fibrin-based sealants. The gelatin-CLOTFOAM adhesive is expected to adhere to lacerated tissue and bind the opposing tissues together with a strength that is significantly higher than that observed for fibrin sealants. The following laboratory tests were conducted interactively with animal experiments (rat, rabbit and swine model for grade III and IV liver wounds).

1. Ex-Vivo Experiments on Baseline Formulation: Adhesion and Coagulation Properties

To determine the feasibility of using CLOTFOAM for intracravitary hemorrhage, we first conducted adhesion and tensile measurements (Intratissular adherence and clot strength) in Sprague-Dawley rats liver tissue. The liver was chosen because is the most frequently damaged organ in intraperitoneal trauma followed by the spleen.

Experimental Models: Sprague-Dawley rats (250 to 300 g) were anesthetized. The abdominal cavity was approached medially and the liver was completely dissected out and excised. The liver was chosen because it is the most frequently damaged organ in intraperitoneal non-compressible hemorrhage followed by the spleen. We conducted adhesion and tensile studies with an isometric transducer.

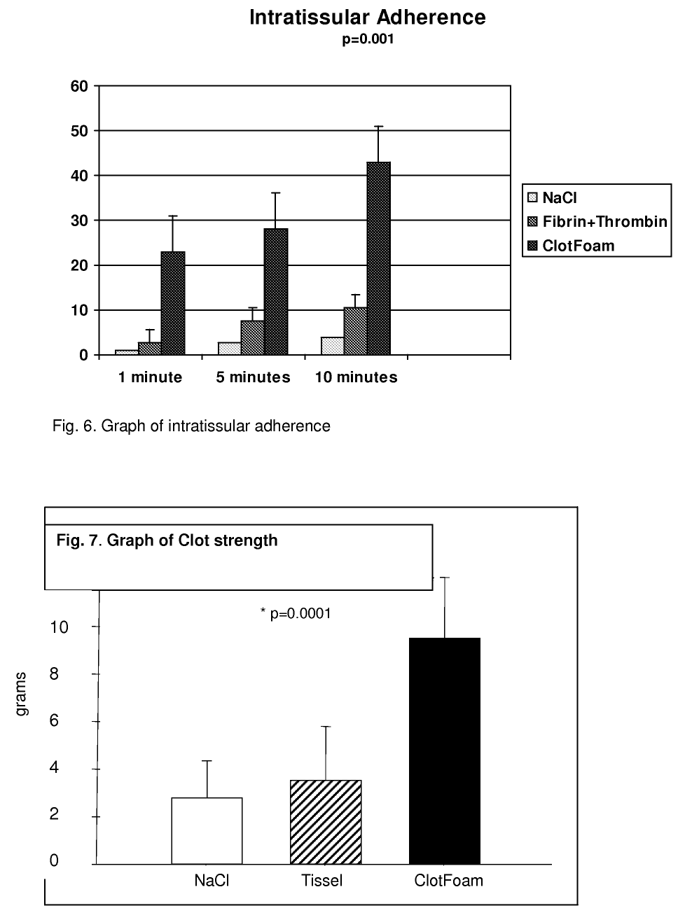

Tensil Measurements: The two largest lobes separated. One lobe was attached to a holder that was fixed later to the isometric transducer. The other lobe was placed in a flat bed of gauze in a container that could gradually be elevated and lowered to produce contact with the piece of liver in the transducer's holder. A damage area of 1 cm² was produced in both liver pieces. The formulation to be tested for tissue adherence was deposited between the two pieces. The specimens were placed in contact at a baseline pressure of 0 gr. At various time points (1, 5 and 10 minutes of exposure and contact), the pressure needed to completely separate them was recorded. We tested the baseline formulation and compared these results with a solution of NaCl as controls. The results of the intratissular adherence are depicted in FIG. 5. The force of adherence induced by ClotFoam after 10 min is more than 200% stronger than the control in the intratissular adhesion secondary to the exposition of damaged tissue to the foam. (FIG. 6)

2. Quantification of Clot Strength:

To study the strength of the formed cloth under the influence of the CLOTFOAM we used the following experimental model: Blood was collected in a test tube previously prepared to contain a strand of cotton suture with a piece of cotton gauze as weight in one end, to maintain that side on the bottom of the tube, and at the other end out of the tube with a loop to hang the strand to the isometric transducer. The strand of cotton suture was included in the blood and allowed to coagulate for 2 minutes. The other end of the strand was fixed to an isometric transducer and then pulled down to measure the force (in grams) necessary to pull up the strand from the clot and test tube.

Strength of the coagulated blood showed statistically significant differences when the two groups were compared using Student's T test (n=6 per group) (FIG. 7). Clot strength can be observed in the three groups blood plus saline solution (B+S), blood alone (BA) and blood with the CLOTFOAM (B+Gel). There is a statistically significant (P=0.001) difference when the blood was treated with CLOTFOAM as compared to blood alone or blood plus saline.

3. Foam Volume Expansion

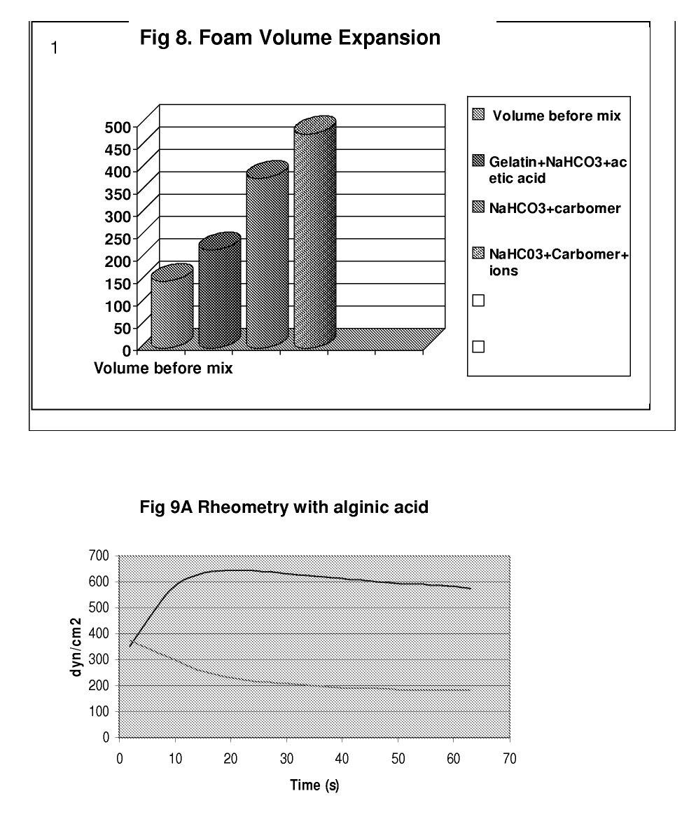

Methods: An analytical method for determination of foam volume expansion was devised and validated. Alteration of the quantity/volume of foam was achieved with the addition of new components—metalic ions and acids—to the formulation and by alterations of the initial pH values of the two constituents. This was done while at the same time maintaining the final pH=7.4 of the mixture. The formulation obtained by modifying concentration of current components and pH and volume varies from 400% to 600% expansion depending on the type of acidic agent used (Carbomer versus Alginic acid) FIG. 8.

The formulation incorporates biocompatible agents that produce foam both by means of chemical reaction and to some degree in response to CO₂ propelled delivery. The foam producing approach is harmless and avoids putting tissues in contact in microenvironments with solutions that breach the extremes of physiological pH that gives rise to undesirable irritation and adhesions. Among the agents that enhance foam upon mechanical agitation several ionic surfactants including Tween 80, sodium lauryl sulfate, sodium lauroyl sarcosinate, taurate salts have been tested. Among the chemical reactants that produce CO₂ several organic acids, polysaccharides and particularly alginic acid, and carbomer were tested. It must be noted that the acid foam producing component simultaneously plays an important role in the cross-linking gelation process.

Protocol: In order to compare inherent foaming capacity, agents were combined with all components of the current formulation (i.e., sucrose, PVP and fibrinogen) with the addition of phosphate buffered saline (pH 7.4), The foaming capacity was quantified by measuring bulk volume, after polymerization, of a known weight of reactants, as displacement by gel in a volume of inert solvent such as hexane or CCl₄. This value was compared to a control formulation, based on bicarbonate and acetic acid.

Additional investigations involved the incorporation of surfactants and additional components to improve volume expansion and decrease gelling time. Experiments of foam expansion were investigated in relation with concentration and adhesive properties. These aims were iteratively pursued, as optimization for a single property my come at the expense of others.

Methods: Each sample was tested 3 times. 1 mL of solution A was simultaneously added to 1 mL of solution B in a 16×100 mm test tube using two 1 mL disposable BD syringes. Each mixture was vortexed for 5 seconds immediately after mixing. Displacement tests were preformed 20 minutes after vortexing and were tested using the highest point of foaming marked by a sharpie on the test tube. The test tube was lowered into a 50 mL graduated cylinder containing 40 mL of acetone until the bottom of the meniscus of the acetone was aligned with the sharpie mark and the displacement in mL was noted. Stickiness tests were performed 40 minutes by shear test after vortexing to ensure the solutions had reached their heightened adhesiveness. Adhesiveness was measured on the pascal scale and values were categorized from 0 to 10, 0 being as adhesive as water and 10 being most adhesive. All tests were performed at 37° C. Adhesiveness gelling time and foam expansion significantly improved upon adding alginic acid and carbomeer.

4. Use of Divalent Metal Ions Prototype ClotFoam to Improve Gel Strength and Foam Volume Expansion

The effect of Ca²⁺, Zn²⁺ and Mg²⁺ on effects on gel strength and with favorable foaming capacity, were investigated. It has been established that these ions could markedly accelerate gelatin crosslinking, fibrin polymerization, and both enhance the length and strength of fibrin filaments as well as the gel strength.

Characterization of the kinetics is of utility as it may indicate that though a given metal ion addend could enhance gel strength, and drastically decrease cross-linking time of the various polymers included in the 3_D structure. The change in gel strength as a function of time was testes first in the absence of divalent metal ions to establish baselines; followed by adding metals ions at various concentrations that produce maximum effects on clot strength.

Concentration of 20 uM and less of Zn²⁺ do indeed decrease the time to clot, as assayed by a simple method devised, but only by 25% at the highest concentration. Currently higher concentrations, but sub millimolar, are being investigated. The addition on Zn enhanced the adhesiveness of the scaffold.

Ion testing with Magnesium Chloride and Zinc Chloride—Solutions were made using the original baseline formulation. Ions were added as follows: 20 μM ZnCl2 solution, a 20 μM MgCl2 solution, a 40 μM ZnCl2 solution, a 40 μM MgCl2 solution, a 60 μM ZnCl2 solution, and finally a 60 μM MgCl2 solution by adding 0.002 M ZnCl2 and 0.002 MgCl2 solutions. Each test for adhesiveness and displacement were performed using a 50 mL graduated cylinder and latex gloves.

The 20 μM MgCl2 solution was the only formulation that showed improvement in volume displacement and adhesive strength. Increasing the concentration for either ion to 60 μM for reduced its foaming ability.

5. Viscoelastic Properties Amid Gelation

The proposed adhesive uses gelatin as the “structural” protein that serves as a scaffold for the fibrin network. ClotFoam's ability to stop bleeding is achieved through the in situ generation of a three-dimensional polymeric network (in situ crosslinking of preformed polymers) that bonds to the tissue, seals the wound with a fibrin clot and stimulates the coagulatory cascade.

The viscoelastic properties of the cross-linked polymer pairs forming a gel are critical to the ability of the agent to resist the flow of blood and attach to the lacerated tissue. Efforts to optimize this properties include a) cross-linking of gelatin with polysaccharides that can form strong gels and firmly bond to soft tissue (e.g.,alginic acid, Poly-(L-glutamic acid, Hyaluronic acid, Carbomer 934); B) the use of alternative materials (e.g., albumin, poly(ethylene oxide)s, albumin, PVP), c) better crosslinking chemistries (e.g., glutaraldehyde, carbodiimide, Calcium-independent transglutaminase enzyme) and d) more controllable polymerization reactions.

Methods: There are two challenges to characterizing the mechanical properties of such hydrogels. First, when such an adhesive “sets”, its bulk mechanical properties undergo dramatic changes and these changes are not readily observed using a single measurement method. Typically, “setting” is initiated by mixing the structural and crosslinking components. As the three-dimensional polymeric network is being formed, the sample undergoes a characteristic sol-gel transition that yields an elastic gel. Visual observation was initially used to characterize gel time, strength and stickiness of the gel. Those formulations that formed strong gels within 20 second of mixing were subjected to rehometry testing to examine the sol-gel transition (i.e., to measure gel time), monitor the initial evolution of mechanical properties, and evaluate the properties of weak gels. To characterize the mechanical properties (adhesive strength and stickness). of “cured” gels we used standard tensile tests and dynamic mechanical analysis.

Rheology: Rheological studies were performed to evaluate the viscoelastic profiles of ClotFoam scaffold formulations amid gelation. All rheological measurements were determined using a RheoStress 600 rheometer (Thermo) following established methodology. Gelation studies were conducted with a parallel plate geometry; all samples were transferred immediately after mixing (time t=0), and measurement started at t=6 s. ClotFoam was first subjected to frequency scanning from 1 to 100 rad/s at 2% strain for 80 loops with 13.55 s under a constant temperature of 37° C. Consequently, for time and stress sweeping tests, storage moduli (G′) and loss moduli (G″) were monitored as a function of time at a 5 Hz frequency and a 2% stress strain at 37° C.

The gelation kinetics and morphological evolution that is considered optimal for this application is rheologically described by a) the intersection of G′ and G″ (crosslinking and change of state from liquid to gel) within 10 seconds following the mixing of part A and B; b) the rapid increase in the value of G′ over 1000 dyn/cm2 pointing to a strong gel; c) maintenance of a high value of G′ for over 10 minutes and decrease of G″ after 10 minutes that return the agent to a liquid state facilitating its absorption by the cavity fluids, and a tangent value increasing from 0.1 to 0.4 indicating an increasing storage energy over released energy.

The above described viscoelastic characters of the foam are expected to provide an improved matrix structure for the developing fibrin network that binds sundered tissue and an effective barrier to blood flow.

Rheological measurements were conducted for the following compositions:
1. Pair of structural polymers
   a) Gelatin, alginic acid
   b) Gelatin, Hyaluronic acid
   c) Gelatin, Poly(L-glutamic acid)
   d) Chitosan, alginic acid
   e) Gelatin, Carbomer 934
2. The following crosslinking catalysers
   a) Calcium-independent transglutaminase enzyme mTg
   b) EDC
3. Concentration of surfactants
4. Alternative materials:
   a) Carboxymethyl Cellulose
   b) Acrylates
5. Dilutions

Use of Rheometers and Clarifications:

Because of a technical malfunction two different rheometers were used to measure the torque required to twist the parallel plate in two different parameters. The first measurements were registered in pascals, while the second rheometer measured in dyn/cm². The conversion factor for this is as follows: 1 Pa=10 dyn/cm2. Experiments were conducted at 37 degrees Celcius. Since the gel point of the compound (where G′ and G″ cross) occurred before the rheometer could take measurements (delay of 10 s.) several different dilutions were performed In order slow down this reaction. A 60% dilution slowed down the reaction enough to determine the gelling in the graph as well as the evolution of the gel strength. Because viscosity is a practical issue in the application through syringe, a 80% dilution was also tested.

Studies on the Replacement of Organic Acid

Alginic acid was substituted by Poly(L-glutamic) acid, hyaluronic acid and carbomer. Rehometry of formulations containing alginic acid and carbomer are compared in FIG. 9. This measurement establishes the improvement of viscoelastic properties when alginic acid is replaced by carbomer.

Studies on Crosslinking Catalysts for Gelatin 3-D Scaffold

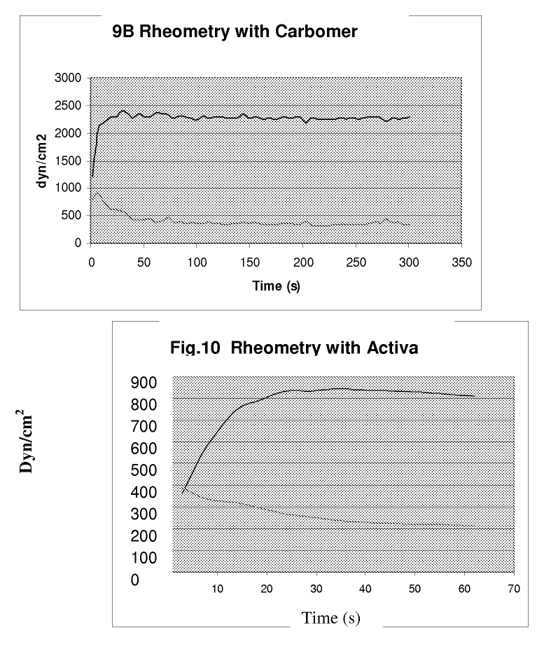

In order to increase the Gelling time and gel strength, cross linking catalysts for the structutral polymers (e.g. Gelatin) were introduced to the formulation 1-Ethyl-3-(3-dimethylaminopropyl)carbodiimide HCl (EDC) test—Addition of EDC did not increase volume expansion but did form a stronger gel and more rigid scaffold. Due to the toxicity and limited improvement in viscoelastic properties, the addition of EDC although confirmed the advantages of adding a cross linking catalyst, as discarded. 2-Addition of the Calcium-independent microbial transqlutaminase (mTG; Activa) test—Four solutions were prepared in this experiment to compare EDC to Activa. Four 5 mL aliquots of Solution B were prepared, adding 0.5 grams and 1 gr. Of EDC and active respectively to each solution. The 0.5 gram Activa solution formed a final product that was more rigid and stronger than the 1.0 gram EDC solution. The formulation containing Activa was subjected to rheometry testing. The calcium-independent microbial transglutaminase (mTG; Activa TI) obtained from Ajinomoto was used without further purification. Rheometry of formulation with Activa at various dilutions are shown in FIG. 10,

9. Rheological Studies on the Effects of Surfactants in the Viscoelastic Behavior of ClotFoam

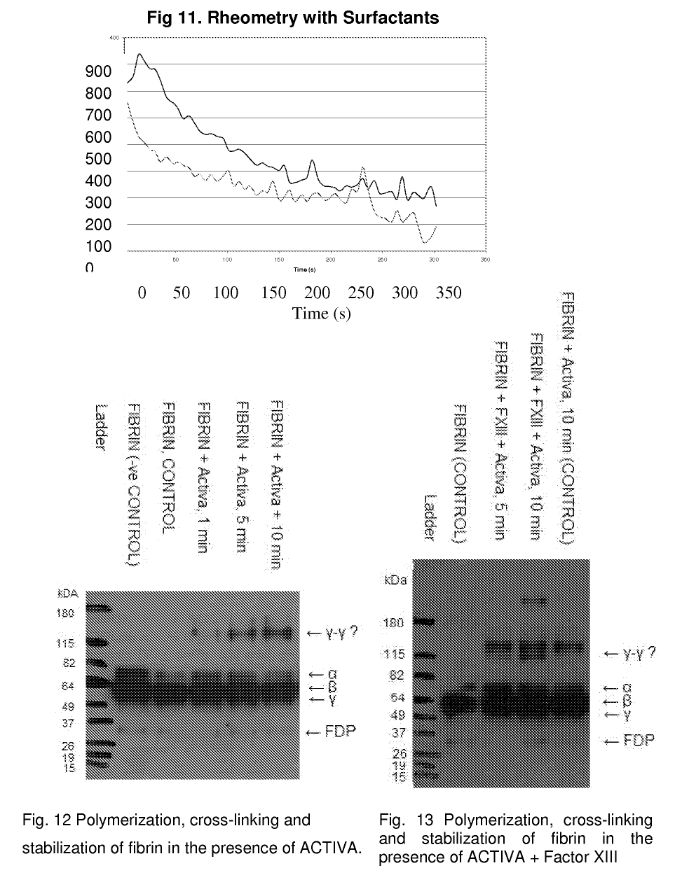

Surfactant Tween 80 was added in several concentrations to the formulation and tested at different dilutions. It is clear from rheometry measurements that surfactants contribute to the reduction of the foam after dispersion but also affect the stability of gel. Al higher dilutions gels break faster. The following formulations tested included Activa and alginic acid at optimal concentrations. Results are shown on FIG. 11.

Rheological measurement show that elimination of surfactant from the formulation reduces the decline in gel strength, thus extending the transition gel-sol.

Summary of the Formulation Process for the Production of an Optimal Scaffold*

Each of the following formulations represent a step forward in regard to optimal viscoelastic properties of the scaffold and fibrin polymerization time for the application.

1) Formulation 1 is an adaptation of the original baseline formulation. The amount of carrageenan in part B was transferred to part A at a 200% concentration. Also, alginic acid and surfactant was incorporated to B.

2) Formulation #2 is similar to formulation 1 except for the addition of EDC and a 10 fold increase in calcium, since EDC is a calcium dependent catalyst.

3) Formulation #3 is an improvement upon formulation 2 in which EDC was replaced with Activa.

4) Formula #7 adds Carboxymethyl Cellulose. The addition of this compound, experimentally proven through visual and rheometric techniques, increased the strength of the gel and slowed down it conversion into the sol state.

5) In formulation #8 Alginic acid and Carboxymethylcellulose are replaced by carbomer 934 which significantly increased the strength of the gel.

6) Formulation #9 is formed by 3 parts in order to maintain Thrombin at neutral pH while fibrinogen remains in Part A at a basic pH and Carbomer in Part 3 at an acid pH. Formulation 9 includes the optimal concentrations of fribrinogen, Thrombin, Factor XIII and fibronectin obtained through western blot assays of the fibrinogen polymerization and fibrin formation (see bellow).

7. Formulation #10 replaces the in situ polymerization of fibrinogen by thrombin with fibrin monomer in acid solution.

9. Molecular Chemistry of Fibrin Polymerization within the Gel

We conducted molecular chemistry assays to compare the effectiveness of Fibrin monomer polymerization (pH Neutralization) and stabilization (cross-linking) by Factor XIII within the scaffold versus fibrinogen polymerization by Thrombin.

9.1. Studies to Determine the Catalyzing Effect of ACTIVA on Fibrin Stabilization

It is well established that FXIII in presence of Ca2+ catalyzed fibrin monomer conversion to tough insoluble fibrin clot. However, weather the presence transglutaminase in the reaction mixture catalyzes crosslinking of free fibrin momomer is not established. Nor has been established the synergic effect of ACTIVA and Factor XIII. In order to follow these reactions, fibrin monomer was subjected to ACTIVA treatment first as a concentration dependent reaction and later a time dependant reaction.

Concentration-dependant and Time-dependant monitored reaction (1, 5, 10 min, respectively), To a volume of acidic solution of 2 mg/ml fibrin was quickly added Activa in 60 mM Tris buffer (pH 8.4, w/2 mM CaCl2) in variable concentration (1.0-20.0 U/ml) to neutralized. The samples in each lane were incubated for 10 min at 37° C. The samples was electrophoresed and transferred to nitrocellulose membrane. The Fibrin was visualized with anti-fibrinogen antibody (1:50). As expected upon quick neutralization of fibrin with buffer generated a number of cross-linked fibrin molecules (lanes 2, 3, 5) with increased concentration of Activa in it when compared with lane 1 containing control sample of fibrin. Furthermore, fragmented derivative products (FDP) of lower kDA bands also participate in crosslinking. High molecular dimmers trtramers

Assays compared a) fibrin and fibrinogen crosslinking by ACTIVA at 1, 5 and 10 Minutes (FIG. 12); b) fibrin and fibrinogen crosslinking by ACTIVA at concentrations of 20 u/ml, 19 U/ml, and 1 U/ml. (FIG. 13);

FIGS. 12 and 13 show the formation of strong gamma dimmers during fibrin cross-linking with ACTIVA and factor XIII at minute 1. At this time gamma dimmers are not yet present in the fibrinogen sample.

9.2 Polymerization, Cross-Linking and Stabilization of Fibrin Within the Scaffold

To test the polymerization rate of the fibrin monomer neutralized by components of the scaffold (PART A and B) and to compare it with fibrinogen polymerization by Thrombin, we conducted western blot essays using anti-fibrinogen antibody. The chains of fibrin polymers were detected by the reaction with polyclonal sheep anti-Human Fibrinogen (Fg) affinity purified peroxidase conjugated antibody. (Cat #: SAFG-APHRP, Enzyme Research Laboratory, IN) for 1 hr (1 part in 50K 5% milk in TBST).

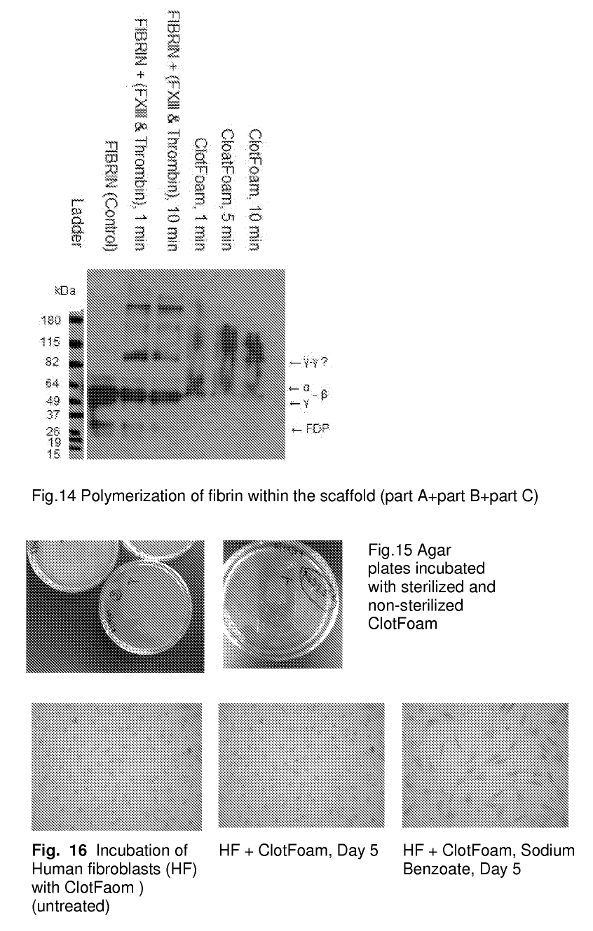

The polymerization of fibrin when mixed to A and B components of ClotFoam is established by the western blot essay shown in FIG. 14.

10. Sterilization

Sterile preparations of clot foam were studied. The Neutral Half of PART A was sterile filtered in a biological safety cabinet using a Nalg-Nunc 500 mL device (Cat #450-0045, nitrocellulose membrane, 0.45 m filter).

The basic PART A: BSA, MgCl₂ and CaCl2 were dissolved in sterile water were added 0.15 g solution. The solution was sterile filter using 0.22 μm Millpore Syringe filter into above 8 mL of Neutral part. To this mixture then was added premeasured and autoclaved sterile 1.2 g of solid NaHCO3 and 0.4 g of Carrageenan.

PART B: sodium monophosphate, tris(hydroxymethyl)aminomethane (TRIS-Base) and Activa were dissolved in sterile water. The solution was sterile filter using 0.22 μm Millpore Syringe filter. To this sterile mixture, 0.5 g UV sterile Carbomer was added

PART C: The acidic Fibrin Monomer was sterile filtered in a biological safety cabinet using a Nalg-Nunc 500 mL device (Cat #450-0045, nitrocellulose membrane, 0.45 m filter).

Growth Study: The general experimental protocol included preparation of sample solutions which were then plated on Potato dextrose agar (PDA, Sigma-Aldrich, Cat #P2182) and Tryptic soy agar (TSA, Sigma-Aldrich, Cat #T4536) gels in Petri dishes for growth. The PDA and TSA gels were incubated and observed at the indicated periods of time for colony growth (mold and/or bacteria).

The sample was incubated for 30 min at 37° C. and evaluated for colony growth using the naked eye at the time periods indicated in the Results and Discussion section. The samples were run in duplicate or triplicate with multiple samples indicated with a 1, 2 and 3 designation in data tables. The scale used for evaluation is as follows:

TABLE 2
Colony Count Key

| Symbol | Count |
|---|---|
| − | No visible growth |
| + | 1-199 visible colonies |
| ++ | 200-399 visible colonies |
| +++ | >400 visible colonies |

Table 3 shows the results of studies of microorganism growth analysis on PDA and TSA of the sterile components of FIBRIN_ClotFoam.

TABLE 3
Sterilization Studies by Bacterial Growth on PDA/TSA at 37° C.

| | | Potato Dextrose Agar (PDA) | | | | | | | | | | Tryptic Soy Agar (TSA) | | | | | |
|---|---|---|---|---|---|---|---|---|---|---|---|---|---|---|---|---|---|
| | | Time Elapsed (days) | | | | | | | | | | | | | | | |
| Sample | #s^{$} | 1 | 2 | 3 | 4 | 5 | 6 | 7 | 11 | 1 | 2 | 3 | 4 | 5 | 6 | 7 | 11 |
| Neutral Half* | 1 | | | | − | | | − | − | | | | − | | | − | − |
| A**\hat{ } | 1 | | | | − | | | − | − | | | | − | | | − | − |
| | 2 | | | | − | | | − | − | | | | − | | | − | − |
| | 3 | | | | + | | | − | − | | | | − | | | − | − |
| B**\hat{ } | 1 | | | | − | | | ! | − | | | | − | | | − | − |
| | 2 | | | | − | | | − | − | | | | − | | | − | − |
| | 3 | | | | − | | | − | − | | | | − | | | − | − |
| C** | 1 | | | | − | | | − | − | | | | − | | | − | − |
| (Fibrin/AcOH, pH 3.5) | 2 | | | | − | | | − | − | | | | − | | | − | − |
| | 3 | | | | \! | | | − | − | | | | − | | | − | − |
| Autoclaved NaHCO₃⁺ | 1 | | | | − | | | − | − | | | | − | | | − | − |
| | 2 | | | | − | | | − | − | | | | − | | | − | − |
| | 3 | | | | − | | | − | − | | | | − | | | − | − |
| Autoclaved | 1 | | | | − | | | − | − | | | | − | | | − | − |
| Carrageenan⁺⁺ | 2 | | | | − | | | − | − | | | | − | | | − | − |
| | 3 | | | | − | | | − | − | | | | − | | | − | − |

*Neutral Half is sterile filtered using 0.45 μ filter, stored for a week at 4° C.
**“sterile” A, B, C used for animal studies in SUNY, stored at 4° C. for a week
\hat{ }add 1000 μL of sterile water only in A & B to make “liquid”
⁺Test Sterility of NaHCO3 (used in A) after autoclaving at 121° C. for 20 min, make a saturated solution in sterile water
⁺⁺Test Sterility of Carrageenan (used in A &B) after autoclaving at 121° C. for 20 min, make a solution in sterile water
^{$}Experiments were performed in triplicates except for Neutral Half
! Experimental error

The growth data indicate that Neutral Half and other sterile components yielded no significant growth even after 11 days. Furthermore, the following techniques could be used for sterilization.

TABLE 4

| Components | Autoclave | UV | Other |
|---|---|---|---|
| NaHCO3 | Yes | — | — |
| Carrageenan | Yes | — | — |
| Carbomer | No | Yes | None |

PART A with Sterilization and Preservatives: PART A prepared by modified sterilization method was added sterile additives in different concentration to inhibit growth. These samples were tested for microbial growth on PDA. The growth data are shown in Table 5

TABLE 5
Bacterial Growth Studies in Presence of Preservatives
on PDA Agar plates (3 days)

| | | | | Methyl 4- | |
|---|---|---|---|---|---|
| | | Germaben II | | hydroxybenzoate | |
| | Samples | Lotionecrafter | | Sigma H6654 | |
| Conditions | Frequency | 0.30% | 1% | 0.25% | 1% |
| PART A, Sterile | | | | | |
| At 4° C. | 1 | — | — | — | — |
| | 2 | — | — | — | — |
| At RT, 24 hrs | 1 | — | — | — | — |
| | 2 | — | — | — | — |
| Neutral Half, sterile | | | | | |
| (CONTROL) | | | | | |
| At 4° C. | 1 | — | — | — | — |
| | 2 | — | — | — | — |
| At RT, 24 hrs | 1 | — | — | — | — |
| | 2 | — | — | — | — |

The growth data indicate that after 3 days, methyl 4-hydroxybenzoate and Germaben II provide sterilization/inhibition of growth. Based on this result, PART A containing methyl 4-hydroxybenzoate or Germaben II will be evaluated for gel/clot formation and compared with gel containing no preservatives.

FIG. 15 compares an Agar plate incubated with non sterilized gelatine and with sterilized ClotFoam at day 10.

Conclusion

Adopting a sterile preparation method inhibits growth of contaminants (mold, bacteria) and may provide an acceptable shelf-life of a commercial product. The addition of preservatives displayed inhibition of microbial growth.

11. Biocompatibility

Two ClotFoam preparations—Formulation #10 and F #10+Sodium Benzoate, were prepared under sterile conditions-were tested. These preparations were tested for biocompatibility with human fibroblasts (HF) and human epithelial cells (A549 cell line, ATCC).

Normal human fibroblasts (HFs) were obtained from a commercial source and cultures established in 60 mm tissue culture plates in Dulbecco's modified Eagle's medium supplemented with 10% fetal bovine serum and maintained at 37° C. in a humidified 5% CO₂ atmosphere (CO₂ incubator). Human epithelial cell line A549 was maintained in Minimal Essential Medium supplemented with 10% fetal bovine serum and 2 mM glutamine. When fibroblast and epithelial cell cultures reached subconfluence, control and sodium benzoate ClotFoam preparations were mixed and immediately delivered into individual dishes. The cultures were returned to the CO₂ incubator and examined daily for a total of five days. ClotFoam material and medium was removed from all cultures, and adherent cells were stained with crystal violet (0.1% in 2% ethanol).

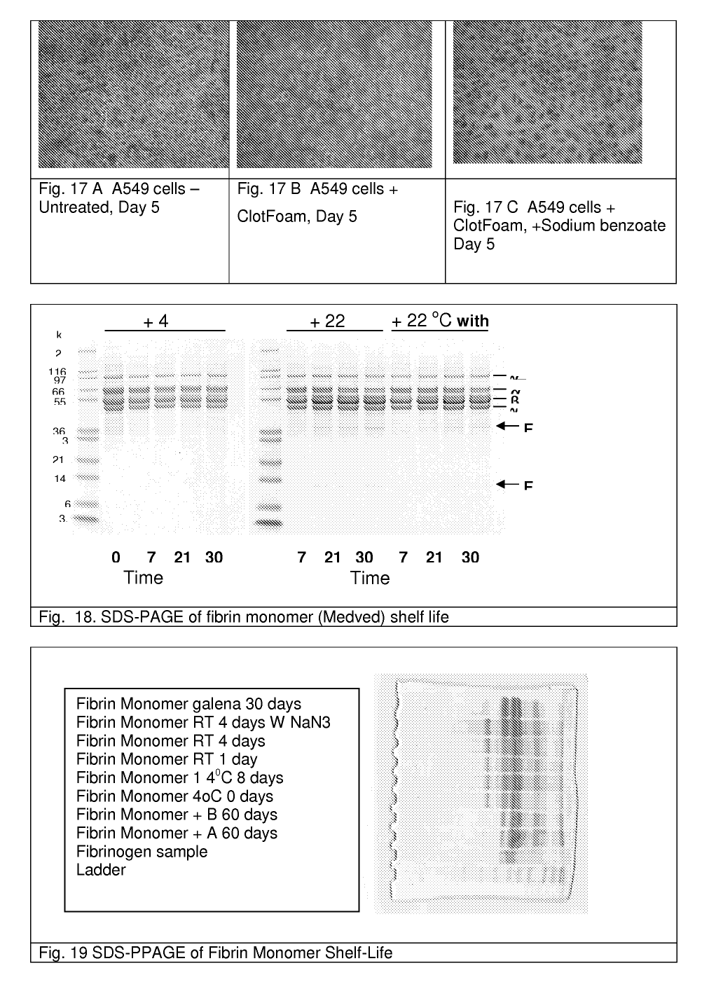

The microphotographs shown in FIG. 16 (HF) and FIG. 17 (A549) illustrate the results. The main observation was a total absence of damage or toxicity to the cells, and absence of any bacterial or fungal contamination. In human fibroblast cultures exposed to ClotFoam preparations, the cells appeared slightly larger or more spread out than in control untreated cultures. Conclusion: ClotFoam and ClotFoam+preservative benzoate are biocompatible, and do not affect, but rather stimulate, the growth and differentiation of cells; which is an important attribute in wound healing agents.

12. Shelf Life

21.1 Shelf Life of Monomer Part C

The purpose of these experiments was to establish that monomer does not degrade over time and under standard conditions (4° C. and 22° C.).

Fibrin monomer was prepared by dialysis of fibrin polymer against 1 liter of 0.125% acetic acid for 20 h with two changes of the dialysis solution (each in 1 h). Fibrin monomer was concentrated to 17.6 mg/ml. Final yield—80%. Fibrin monomer was then divided into 3 portions, one was kept at 4° C., the other two, one of which contained sodium azide, were kept at room temperature (22° C.); sample for the analysis have been withdrawn at the indicated time.

The shelf life of Fibrin monomer was analyzed by SDS-PAGE and stained with Imperial Protein Stain (ThermoScientific). The analyses of these samples were performed after 1, 4 and 15 days and compared with the Fibrin monomer samples obtained by independent method (Medved & Oglev, unpublished data) and stored for over 30 days at 4° C. FIG. 18 and FIG. 19.

Clotability

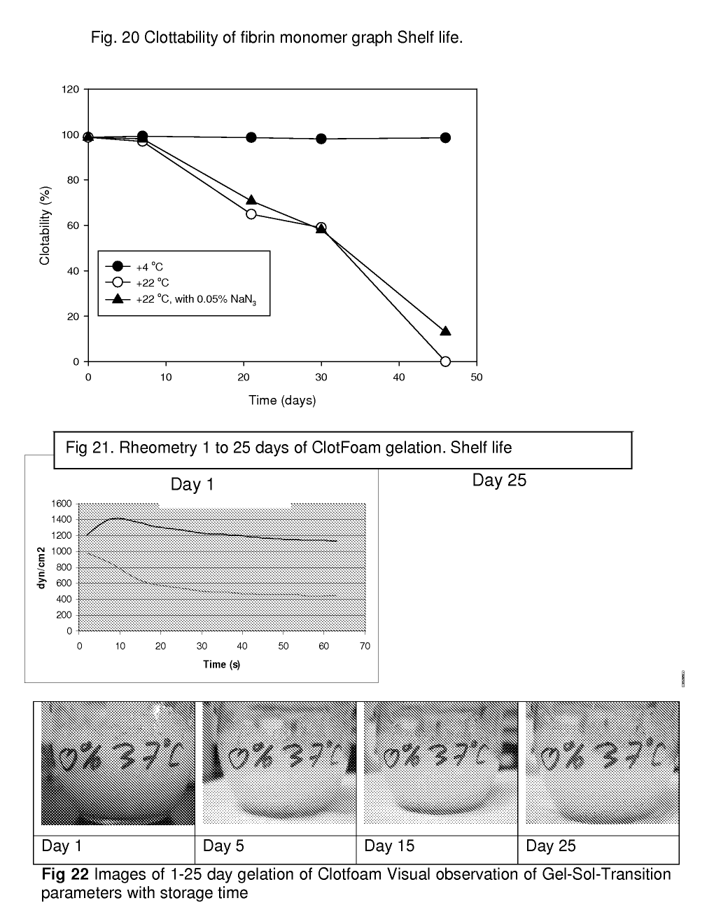

DSPage data show that there no visible fibrin degradation over time neither at 4)C or 220 C. Clotability studies by the absorption method (FIG. 20) shows that the monomer maintain stability over 6 weeks, while at 220 C stability is maintained over week. Conclusion the Fibrin Monomer produced by either method described in the this patent can be stored at 4° C. for over 60 days.

Shelf Life of Scaffold Part A and B

The shelf life of the scaffold was tested by measuring the strength of the gel produced upon mixing parts A and B and the gel-sol transition period over storage time. The strength of the compound over the course of 25 days was observed via rheometry. (FIG. 21)—A series of images were produced to visually represent the break down of the compound over time at various temperatures (FIG. 22).

13. Experiments in Animal Models

We conducted studies on a simulated intracavitary trauma on rats and swine (pig) models.

Effect of Baseline ClotFoam on Blood Loss After Grade III Liver Injury (Rat Models)

Methods: Ten male Sprague-Dawley rats (225-250 g) were used (approved by the Institutional Animal Care and Use Committee of UMB).

Experimental Procedure: A laparotomy was performed; Grade III liver injuries were induced in the larger left and right lobes. The injury was induced by clamping with a hemostatic clamp on both lobules and causing injury through the parenquima of the liver of the two medial lobes.

After the first penetration of the liver, the clamp was opened and repositioned to the animal's left inducing the second lesion including more than 40% of distance from the border to the suprahepatic vena cava. After this repositioning, the liver was penetrated a second time. Further documentation of the liver injury was achieved by excision and inspection of the liver at the conclusion of the experiment. The injuries were through and through. No concomitant damage to the common bile duct, caudal vena cava, or hepatic artery was noted. Ten animals received injuries for this study, and they were assigned randomly to receive either 4 ml of saline solution (Control) or 4 ml of ClotFoam agent. Immediately after the injury was induced, ClotFoam or saline was administered through a needle into the peritoneal cavity.

Results: All injuries were through and through, with no major differences noted among treatments. Bleeding time in control group showed a mean of 37 seconds (±SD 7.5) while the amount of blood allows the observation. In contrast, the CLOTFOAM (HAF) treated group stops almost immediately the bleeding of injured areas with 4 seconds (+1.0).

Blood loss measurements in the control untreated group were 2.45 mL (from 1.86-3.61) with a SD of 0.67 in the control group in contrast the treated group had a mean of 1.45 mL (0.72-2.08) with a SD of 0.55, T-test showed this difference to be statistically significant with a p value of 0.028.

Validation of Efficacy of baseline formulation in Liver Damage (Rat Models)

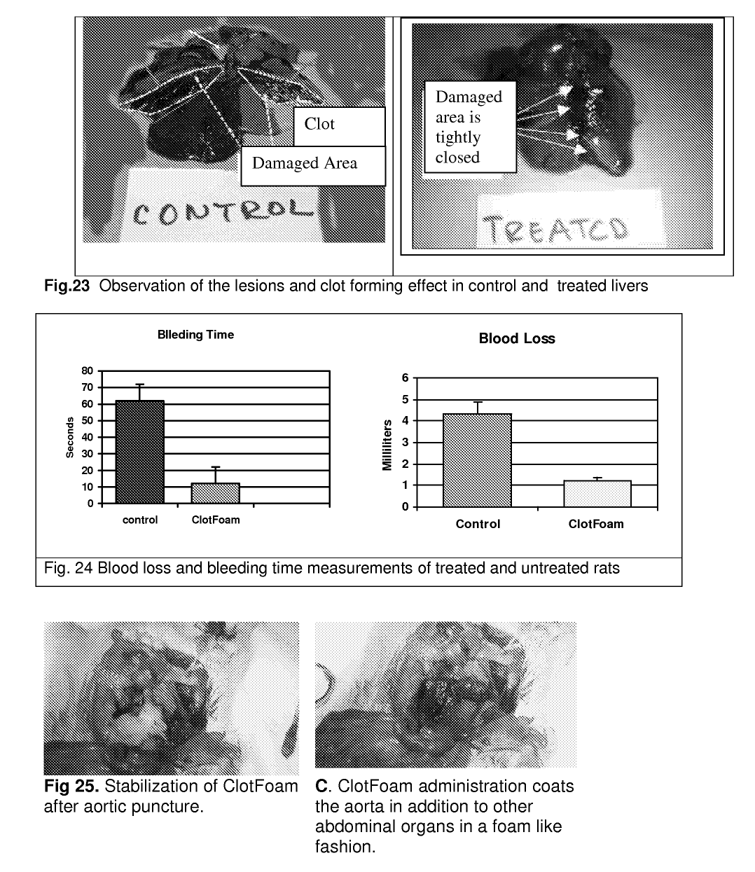

Livers were removed for observation of the lesions and clot forming behavior. It was found that in controls the damaged areas develop some clots on them but invariably they remain separated (FIG. 23). In contrast, when the ClotFoam is administered, livers show the formation of very strong clots in the injured areas, with no adherence of the clot to the undamaged tissue.

Effect of ClotFoam on Blood Loss After Grade IV Liver Injury in Rats.

Methods: Ten male Sprague-Dawley rats weighing 225-250 gr. were used in this study. Laparotomy was performed, Grade IV liver injuries were induced in the larger left and right lobes. The injury was induced by clamping with an hemostatic clamp both lobules and causing injury through the parenquima of the liver and underlying vessels of the two medial lobes. After the first penetration of the liver, the clamp was opened and repositioned to the animal's left inducing the second lesion including more than 80% of distance from the border to the suprahepatic vena cava. After this repositioning, the liver was penetrated a second time. Further documentation of the liver injury was achieved by excision and inspection of the liver at the conclusion of the experimental period. The injuries were through and through, with one or more of the left medial lobar vein, right medial lobar vein, and portal hepatic vein lacerated. No concomitant damage to the common bile duct, caudal vena cava, or hepatic artery was noted.

Animals were assigned randomly to receive either saline solution (Control) 4 ml or the ClotFoam agent 4 ml. Immediately after the injury was induced, the treatement or saline was administred through a needle in the peritoneal cavity.

The bleeding time was observed and recorded. Then the abdominal cavity was closed with 4-0 nylon to observe the animals for 90 minutes. After this period of time the animal was re-anesthetized and all the fluids in the abdominal cavity were collected in a pre weighted gauze and re weighted to measured the intraperitoneal volume and calculate blood losses.

Results

All injuries were through and through, and major vessels lacerated with no differences noted among treatments. Bleeding time in control group showed a mean of 37 seconds (±SD 7.5) while the amount of blood allows the observation. In contrast, the CLOTFOAM treated group stops almost immediately the bleeding of injured areas with 4 seconds (+1.0). This difference showed statistical significance with p<0.008.

Blood loss measurements in the control untreated group were 2.45 mL ( from 1.86-3.61) with a SD of 0.67 in the control group in contrast the treated group had a mean of 0.95 mL (0.72-1.78) with a SD of 0.55, T-test showed this difference to be statistically significant with a p value of 0.028 (FIG. 24).

Aortic Model in Close and Open Cavity Experiments

Methods: In this model, a midline laparotomy is made. The aorta is clamped just below the renal arteries and just above the bifurcation of the iliac arteries, effectively gaining infrarenal proximal and distal aortic control. The infrarenal aorta is then pierced with a 25 gauge needle once on both left and right sides of the vessel. After 6 seconds of uncontrolled bleeding, 500 microliters of ClotFoam is applied diffusely throughout the intraperitoneal cavity. After completion of foam application, time to hemostasis is measured. The abdomen is then closed.

Immediately after injury, the rat is given Ringer's solution to maintain mean arterial pressure at about 70-80% of initial MAP (if possible) which is the current standard resuscitation technique for trauma patients. The rat is observed for 20 minutes. After 20 minutes, the animal is re-explored through the same midline incision. All of the blood is collected with pre-weighed gauze pads and total blood loss is calculated (FIG. 25).

Results: Seventeen animals underwent aortic injury. Animals were randomized into 2 different groups: treated and non-treated with ClotFoam. Survival was 100% at 60 minutes for all animals treated with ClotFoam. No animals survived the injury in the no treatment group. All pre-injury MAP were similar. Table 1 below summarizes the outcomes measured in each group.

TABLE 1
Comparison of outcomes for aortic-injured animals with three different
ClotFoam formulations and those without treatment: Resuscitation
index is defined as the resuscitation MAP percentage of pre-injury MAP.

| Table 6 | Form #10 | No agent |
|---|---|---|
| Outcome | (N = 10) | (N = 7) |
| Time to hemostasis (s) | 12.2 ± 2.9 | N/A |
| Total blood loss (ml) | 5.2 ± 0.5 | 16.3 ± 0.3 |
| Resuscitation index (%) | 68.8 ± 14.0 | 26.8 ± 2.4 |
| Resuscitation volume (ml) | 12 ± 4.5 | 20.3 ± 2.5 |
| Survival (min) | 60 ± 0 | 18..3 ± 2.9 |

P values for all outcomes and all formulations are <0.001 compared to no agent.

Cava Vein Model

The second model is a liver/vena caval injury model. In this model, a small upper midline laparotomy is made. The left lobe of the liver and the vena cava are exposed and isolated. A small incision is created in the right lower quadrant and the ClotFoam applicator tip is placed through that incision so that the opening is intraperitoneal but remote from where the injury will take place. Next the injury is created by sharply transected the left liver lobe and then creating a stab injury into the vena cava. The mini-laparotomy incision is rapidly closed with staples. ClotFoam is then injected into the closed abdominal cavity. Resuscitation, observation and blood loss measurements are collected as mentioned above.

Twelve animals underwent liver/vena cava vein injuries. Animals were randomized into different two groups: treated and untreated with ClotFoam. Survival was 100% at 60 minutes for all animals treated with ClotFoam.

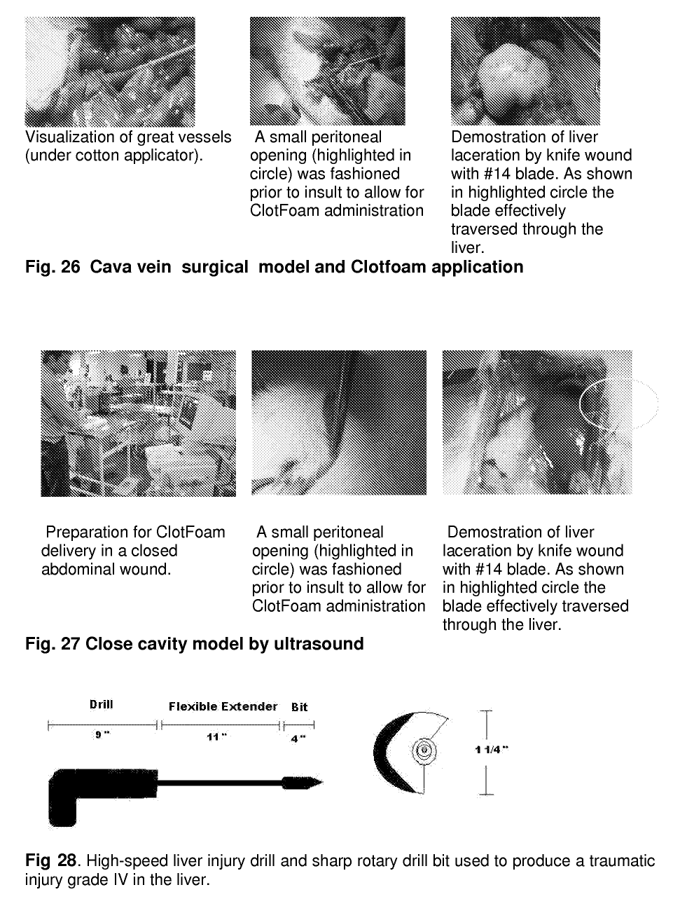

The applicator was placed into the intraperitoneal cavity. At the 20 minute mark after liver/caval injury, the animal was opened fully to expose the injured area. As shown in FIG. 26, ClotFoam is present in the pooled blood of area B, demonstrating its ability to move to target tissue despite blind (and remote) intracavity application.

The animals in the no treatment group died at >18 minutes from injury. All pre-injury MAP were similar. Table 7 summarizes the outcomes measured in each group.

TABLE 7
Comparison of outcomes for liver/cava-injured animals conducted
at UMB with and those without treatment: Resuscitation index is defined
as the resuscitation MAP percentage of pre-injury MAP.

| Table 7. | Form 10 | No agent |
|---|---|---|
| Outcome | (N = 7) | (N = 5) |
| Total blood loss (ml) | 3.6 | 14.7 |
| Resuscitation index (%) | 88.6 | 54.3 |
| Resuscitation volume (ml) | 10 | 16 |
| Survival (min) | 30 | 18 |

Close Cavity Model

Spague Dawley rats were monitored for blood pressure and mean arterial pressure (MAP) via femoral catheterization and A-line placement. In this approach no laparotomy was performed. Instead a #14 blade was used to slice through the upper right abdominal quadrant more closely mimicking a knife wound. The area of wound was determined by ultrasound. The internal Prior to the insult a small incision (0.5-1.0 cm) was placed on the lower left abdomen to allow placement of the delivery apparatus (FIG. 27)) into the abdominal cavity for ClotFoam administration.

Similar drops in blood pressure and MAP occurred after the blade was used to puncture the liver. After 10 seconds ClotFoam was administered through the lower left abdominal port under manual pressure. Once again the blood pressure stabilized and hemostatsis was maintained through 2 hours when animals were sacrificed for analysis.

Summary of ClotFoam effects in three different hemorrhagic models: Data in table 8 compares results obtained with the different models. Control represents puncture model with NS treatment alone. Numbers represent blood pressure and mean arterial pressure (MAP—in parenthesis) at baseline 10 seconds, 10 minutes and two hours post insult. NS—normal saline administration in ml. NA—not applicable (control animals died before 2 end point). TTH—total time to hemostatsis, measured as function of a sustained and maintained rise in blood pressure and MAP of 70.

TABLE 8

| | | | NS | | | | |
|---|---|---|---|---|---|---|---|
| Model | baseline | 10 sec | (ml) | 10 min | 2 hrs | TTH | Survival |
| Aortic | 151/110 | 38/31 | 4.0 | 133/124 | 131/117 | 3 m | all |
| | (120) | (34) | | (110) | (124) | | |
| Venous | 140/105 | 63/34 | 3.5 | 109/69 | 130/72 | 2 m | all |
| | (115) | (41) | | (83) | (97) | | |
| Puncture | 162/102 | 66/41 | 4.2 | 131/69 | 135/87 | 2 m | all |
| | (128) | (50) | | (87) | (102) | | |
| Control | 150/112 | 50/37 | 9.7 | 75/38 | NA | NA | none |
| Venous | (128) | (41) | | (48) | | | |
| Control | 153/108 | 42/34 | 10.5 | N/A | N\A | N/A | none |
| aortic | (119) | (33) | | | | | |

Studies in Pigs

Formulation 10 was evaluated in the pig model.

Methods: Eighteen female Yorkshire crossbred swine, age 2.5 months, weighing 37±2 kg, were used. The protocol was approved by the Institutional Animal Care and Use Committee.

Animals then underwent either grade 3 or 4 liver injuries via open laparotomy or by laparoscopy. For the purposes of this model, a grade 3 injury is defined as a 3 cm long full-thickness parenchymal laceration (created sharply by an 11 blade scalpel). After the liver was exposed, a spot in the middle of the liver was selected to produce the liver injury with a scalpel. The position was calculated by approximation to the suprahepatic vessels and some branches of the portal vein. The spot was marked with a marker. After the damage was induced, surgeons close the cavity, allowed for 30 seconds of massive bleeding before applying ClotFoam through a small perforation.

A grade IV injury was a 10 cm deep parenchymal injury with a specially designed high-speed drill with a cutting drill bit creating an injury akin to a penetrating gunshot (GSW). These injuries were consistent with the American Association for the Surgery of Trauma Organ Injury Scaling system. FIG. 28 describes a high-speed liver injury drill and sharp rotary drill bit used in the experiment.

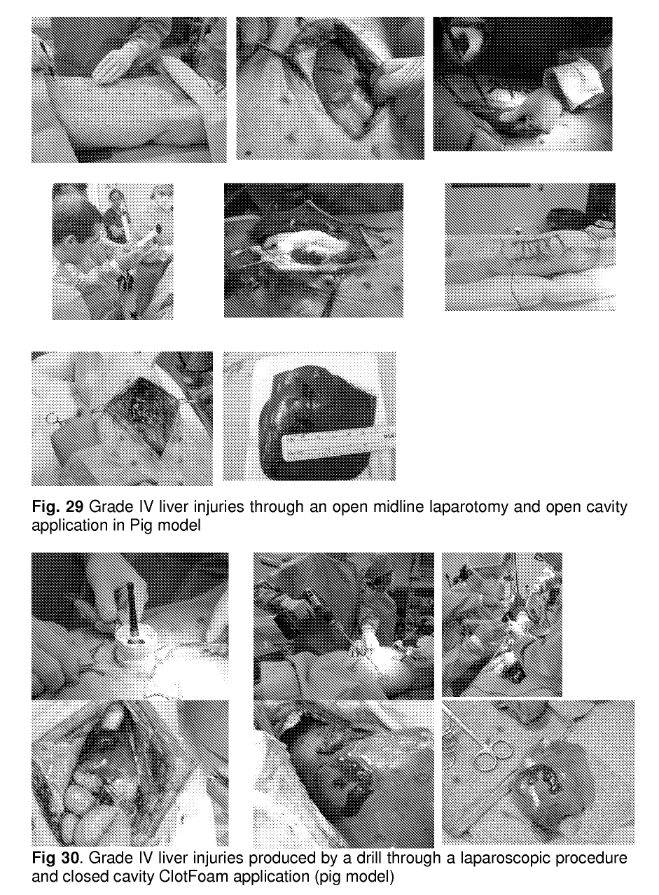

Animals were randomized into 4 groups to date. Group 1 (n=5) consisted of animals who underwent grade IV liver injuries through an open midline laparotomy and had open cavity ClotFoam application (FIG. 29). In this group the agent was visually directed to the liver injury. Group 2 (n=6) consisted of animals who underwent grade IV liver injuries produced by a drill through a laparoscopic procedure and had closed cavity ClotFoam application (FIG. 30). In this group the agent was administered into the peritoneal cavity blindly without direct injury visualization or direction. Group 3 (n=4) consisted of animals underwent grade 4 liver injuries through an open midline laparotomy without ClotFoam treatment (open controls). Group 4 (n=3) underwent grade 4 liver injuries through the laparoscopic technique without ClotFoam treatment (laparoscopic controls).

In all groups, 150 cc of ClotFoam was used for treatment. The ClotFoam was delivered via mixing syringes propelled into the abdominal cavity using pressurized carbon dioxide (approximately 20 psi).

Fluid resuscitation with Lactated Ringer's (LR) was begun immediately after injury. LR was infused as necessary to re-establish a MAP within at least 80% of the pre-injury MAP if possible. Resuscitation was continued for the entire observation period. At the end of the 60 minute study, each animal's MAP and the total resuscitation volume infused were recorded.

After completion of the study period, the abdomen was examined. Liquid blood was suctioned. Blood clots were removed and weighed. In the gauze packing group, additional liquid blood loss was calculated by subtracting the wet gauze weight from dry gauze weight. Total blood loss was determined by adding liquid and clotted blood losses.

Animal survival was defined as the presence of a heart rate at the end of the study period. At 60 minutes, surviving animals were euthanized with 10 ml of Euthasol.

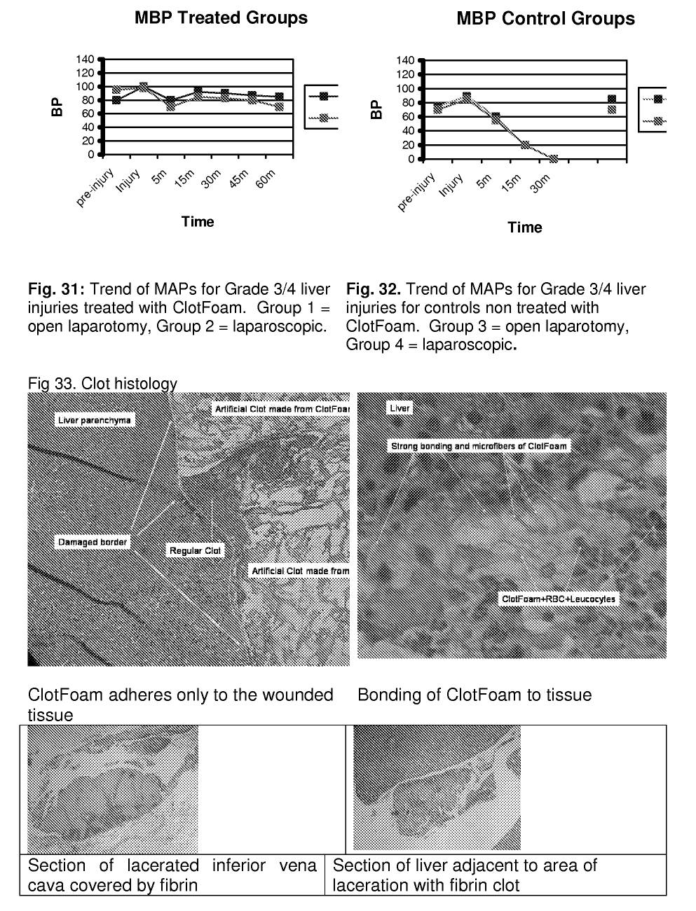

Results: End points for animals in Groups 1 and 2 (Grade IV injuries) are shown in Table 9. Trend of mean arterial pressures (MAPs) are seen in FIG. 31 (treated).

TABLE 9
Outcome measures for Grade 3 liver injuries treated with ClotFoam.

| | | | Fluid |
|---|---|---|---|
| Table 9. | Survival | Total Blood | Requirement |
| Group | Time (min) | Loss (ml) | (ml) |
| 1 (n = 5) | 60 ± 0 | 300 ± 283 | 1500 ± 283 |
| 2 (n = 5) | 60 ± 0 | 600 ± 212 | 2175 ± 742 |

Group 1 = open cavity,
Group 2 = closed cavity.
All values reported as mean ± SEM

Controls: Animals underwent grade IV liver injuries (3 laparoscopic and 4 open) to validate the laparoscopic model against the established open model. These animals were not treated with ClotFoam. Endpoints are seen in Table 10.

TABLE 10
Outcome measures for Grade 3 liver injuries treated with ClotFoam.

| | | | Fluid |
|---|---|---|---|
| | Survival | Total Blood | Requirement |
| Group | Time (min) | Loss (ml) | (ml) |
| 3 (n = 4) | 26 ± 3 | 1900 ± 424 | 3050 ± 70 |
| 4 (n = 4) | 22 ± 11 | 1700 ± 200 | 2467 ± 569 |

Group 3 = open laparotomy,
Group 2 = laparoscopic.
All values reported as mean ± SEM

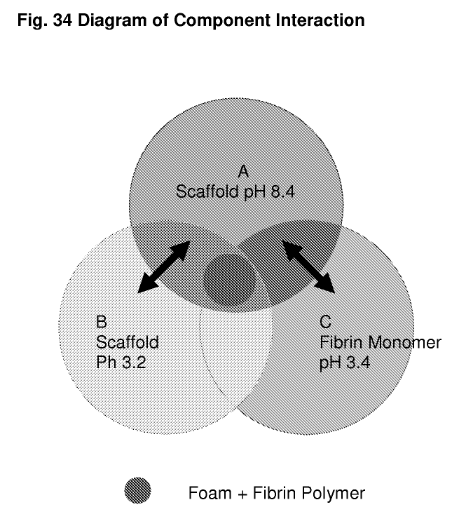

Trend of MAPs for Grade 4 liver injuries of controls untreated with ClotFoam are shown in FIG. 32.

Clot Histology

Liver section samples were collected from all animals at necropsy. Samples of liver, containing the wounded site, were preserved in 5% formalin and processed using standard histology techniques. Fixed tissue samples were embedded in paraffin wax (melting point 56° C.) and sectioned at 2-3 μm. Glass-slide-mounted sections were then stained with hematoxylin and eosin (H&E). Two liver sections perpendicular to the resection site were evaluated per animal. Section are shown in FIGS. 33(a, b, c, d, e, and f)

Conclusion: ClotFoam proved capable of forming a cross-linked foam structure (gel) that rapidly disperses in the intraperitoneal spaces inducing clot formation. When used in non-compresible intraperitoneal hemorrhage secondary to grade IV liver injuries in small animal models, ClotFoam significantly decreases the bleeding time and blood loss, and significantly improves the adhesion between lacerated and damaged tissue. Considering that the total circulating volume in animal models used, the blood loss of ClotFoam treated animals amounts to 8% to 12%, while the controls lost over 25% to 35% of their total blood volume.

### LITERATURE CITED

1. Blood C G, Puyana J C, Pitlyk P J, et al. An assessment of the potential for reducing future combat deaths through medical technologies and training. JTrauma. 53(6):1160-5. 2002.

2. Champion H R, Bellamy R F, Roberts C P, Leppaniemi A. A profile of combat injury. J Trauma. 54:S13-9 2003

3. McManus J G, Wedmore I. Modern Hemostatic Agents for Hemorrhage Control—A Review and Discussion of Use in Current Combat Operations. USArmy Institute of Surgical Research).

4. Deodhar S D, Patel N P, Shah K B, Jammihal H J. Blunt and penetrating abdominal injuries (a study of 51 cases). J Postgrad Med; 29:96-9.1983.

5. Deodhar S D, Patel N P, Shah K B, Jammihal H J. Blunt and penetrating abdominal injuries (a study of 51 cases). 29, 96-99.1983.

6. Deodhar, S. D., Majumdar, H. P. and Agrawal, J. B.: Pattern of assault. Ind. J. Med. Sci., 30: 95-97,1976.

7. Nance, F. C., Wennar, M. H., Johnson, L. W., Ingram, J. C. and Cohn, I. Jr.: Surgical judgement in the management of penetrating wounds of the abdomen Experience with 2212 patients. Ann. Surg., 179: 639-646,1974.

8. Rajdeo, H. P. and Deodhar, S. D.: Stab wounds of the abdomen-A study of 75 cases. Ind. J. Med. Sci., 29: 54-59, 1975.

9. Nance, F. C. and Cohn, I. Jr.: Surgical judgment in the management of stab wounds of the abdomen. A retrospective and prospective analysis based on 600 stabbed patients. Ann. Surg., 170: 569-580, 1969.

10. Nance, F. C., Wennar, M. H., Johnson, L. W., Ingram, J. C. and Cohn, I. Jr.: Surgical judgement in the management of penetrating wounds of the abdomen Experience with 2212 patients. Ann. Surg., 179: 639-646, 1974.

11. Deodhar, S. D. and Parmar, A. R.: Closed injuries of the liver. Ind. J. Med. Sci., 22: 247-250, 1968.

12. Deodhar, S. D., Majumdar, H. P. and Agrawal, J. B.: Pattern of assault. Ind. J. Med. Sci., 30: 95-97, 1976.

13. Schelling G, Block T, Gokel M, Blanke E, Hammer C, Brendel W. Application of a fibrinogen-thrombin-collagen-based hemostatic agent in experimental injuries of liver and spleen. J Trauma. 28:472-5. 1988.

14. Mosher D F, Schad P E. Cross-linking of fibronectin to collagen by blood coagulation Factor XIIIa. J Clin Invest. 64:781-7. 1979.

15. Rousou J, Levitsky S, Gonzalez-Lavin L, Cosgrove D, Magilligan D, Weldon C, Hiebert C, Hess P, Joyce L, Bergsland J, et al. Randomized clinical trial of fibrin sealant in patients undergoing resternotomy or reoperation after cardiac operations. A multicenter study. J Thorac Cardiovasc Surg. 97:194-203. 1989

16. Dunn C J, Goa K L. Fibrin sealant: a review of its use in surgery and endoscopy. Drugs. 58:863-86. 1999

17. Biggs G, ClotFoamron J, Feliciano J, Hoenig D M. Treatment of splenic injury during laparoscopic nephrectomy with BioGlue, a surgical adhesive. Urology. 66:882. 2005.

18. Platelet Protocols. Research and Clinical Laboratory Procedures. McCabe White M. and Jennings L. K. Academic Press, 1999.

19. Azam M, Andrabi S S, Sahr K E, et al. Disruption of the mouse mu-calpain gene reveals an essential role in platelet function. Mol Cell Biol. 21:2213-20, 2001.

20. Hodivala-Dilke K M et al, Beta3-integrin-deficient mice are a model for Glanzmann thrombasthenia showing placental defects and reduced survival. J. Clin. Investig. 103:229-238. 1999.

20. Kjaergard H K, Velada J L, Pedersen J H, Fleron H, Hollingsbee D A. Comparative kinetics of polymerisation of three fibrin sealants and influence on timing of tissue adhesion. Thromb Res. Apr. 15, 2000;98(2):221-8)

21. (Fang N, Zhu A, Chan-Park M B, Chan V. Adhesion contact dynamics of fibroblasts on biomacromolecular surfaces. Macromol Biosci. Oct. 20, 2005;5(10):1022-31.)

22. Endres G F, Scheraga H A. Equilibria in the fibrinogen—fibrin conversion. Polymerization acceptor-modified fibrin monomer. Biochemistry. 1968 December;7(12):4219-26.

23. Action of fibrinogen degradation products and fibrin monomer soluble complexes on platelet aggregation. Larrieu M J. Scand J Haematol Suppl. 1971;13:273-9.

24. Strukova S. Blood coagulation-dependent inflammation. Coagulation-dependent inflammation and inflammation-dependent thrombosis. Front Biosci. Jan. 1, 2006;11:59-80.

25. McDermott M K, Chen T, Williams C M, Markley K M, Payne G F. Mechanical properties of biomimetic tissue adhesive based on the microbial transglutaminase-catalyzed crosslinking of gelatin. Biomacromolecules. 2004 July-August;5(4):1270-9.

26. Francis C W, Marder V J. Increased resistance to plasmic degradation of fibrin with highly crosslinked alpha-polymer chains formed at high factor XIII concentrations. Blood. 1988 May;71 (5):1361-5.

27. Okumura N, Terasawa F, Haneishi A, Fujihara N, Hirota-Kawadobora M, Yamauchi K, Ota H, Lord S T. B:b interactions are essential for polymerization of variant fibrinogens with impaired holes ‘a’. J Thromb Haemost. 2007 December;5(12):2352-9. Epub Oct 8, 2007

28. Hick E J, Morey A F, Harris R A, Morris M S. Gelatin matrix treatment of complex renal injuries in a porcine model. J Urol. 2005 May;173(5):1801-4. J. Biol. Chem., Vol. 269, Issue 37, 23357-23366, 09, 1994

29. Ozeki M, Tabata Y. In vivo degradability of hydrogels prepared from different gelatins by various cross-linking methods. J Biomater Sci Polym Ed. 2005;16(5):549-61.

30. Solgaard G, Haug I J, Draget K I. Proteolytic degradation of cold-water fish gelatin solutions and gels. Int J Biol Macromol. Aug. 15, 2008;43(2):192-7. Epub May 16, 2008.

31 Lekakou C, Lamprou D, Vidyarthi U, Karopoulou E, Zhdan P.

Structural hierarchy of biomimetic materials for tissue engineered vascular and orthopedic grafts. J Biomed Mater Res B Appl Biomater. 2008 May;85(2):461-8.

32. Rocco M. Fibrin formation on fast forward. Blood. May 15, 2008;111(10):4839.

33. Knotzer H, Pajk W, Maier S, Dunser M W, Ulmer H, Schwarz B, Salak N, Hasibeder W R. Comparison of lactated Ringer's, gelatine and blood resuscitation on intestinal oxygen supply and mucosal tissue oxygen tension in haemorrhagic shock. Br J Anaesth. 2006 Oct;97(4):509-16. Epub Aug. 16, 2006.

34. Belitser V A, Varetskaja T V, Malneva G V. Fibrinogen-fibrin interaction. Biochim Biophys Acta. Feb. 19, 1968;154(2):367-75

35. Radzevich I M, Khodorova E L. Preparation of fibrin monomer from fibrin of unfractionated blood plasma] Ukr Biokhim Zh. 1969;41(4):367-70. Ukrainian.

36. Varetskaia T V. Ukr Biokhim Zh. 1965;37(2):194-206.

37. Endres G F, Scheraga H A Equilibria in the fibrinogen—fibrin conversion. Polymerization of acceptor-modified fibrin monomer. Biochemistry. 1968 Dec;7(12):4219-26.

38. Lipiński B. Soluble fibrin monomer complexes] Postepy Biochem. 1968;14(3):373-81. Review. Polish.

39. Copley Al, Luchini Bw. The Binding Of Human Fibrinogen To Native And Fraction Fibrins And The Inhibition Of Polymerization Of A New Human Fibrin Monomer By Fibrinogen. Life Sci. 1964 November;3:1293-305. No Abstract Available.

40. Brass E P, Forman W B, Edwards R V, Lindan O. Fibrin formation: the role of the fibrinogen-fibrin monomer complex. Thromb Haemost. Aug. 31, 1976;36(1):37-48.

41. Lugovsko\check{i} E V, Gogolinskaia G K, Derzskaia S G, Belitser V A. Properties of 2 fibrin monomer species which differ in the degree of thrombin activation. Characteristics of successively appearing active sites] Biokhimiia. 1978 June;43(6):1045-53.

42. Ehrbar M, Rizzi S C, Hlushchuk R, Djonov V, Zisch A H, Hubbell J A, Weber F E, Lutolf M P. Enzymatic formation of modular cell-instructive fibrin analogs for tissue engineering. Biomaterials. 2007 September;28(26):3856-66. Epub Apr. 5, 2007.

What is claimed is:

1. A method of making a scaffold/carrier for a fibrin sealant for use as a non-compressible hemostatic agent that can seal laceraterated tissue and control severe hemorrhage without the need of stiches, or other elements of compression.

2. A method of claim 1 by which the hemostatic agent can be mantained in a 3-part solution.

3. A method of claim 2 wherein 2 solutions (A and B) form a gelatin based scaffold and a third (C) solution carries fibrin monomer.

4. The method of claim 2 wherein the.water-solubilized three-part components of the adhesive can be mixed and polymerized/crosslinked by a polymerization/crosslinking reaction.

5. A method of claim 3 wherein the mixture of the three solutions polymerizes and stabilizes de fibrin monomer thereby forming a fibrin sealant.

6. The method of claim 5 wherein said polymerization of fibrin monomer is produced by means of the acid solution with alkaline PART A being added to said fibrin monomer.

7. A method of claim 4 wherein the solution can be mixed and delivered into the cavity as a foaming gel.

8. The adhesive composition of claim 5 wherein the ratio of the volume of the three mixtures is in a range of 1:1

9. The composion of claim 3 wherein a first container (PART A) maintains in liquid state Teleostan Gelatin type A mixed with sucrose, polyvinylpyrrolidone, bovine serum albumin in buffer solution at a pH 8.3 in the presence of metalixc ions; a second container PART B polymerization/crosslinking agents such as active and Factor XIII, and a high molecular weight polymer such as carbomer in a pH 3.4 solution; and a third container PART C maintains a fibrin monomer in acid solution that polymerizes by a change of pH

10. The composition of claim 9 wherein wherein said alkaline buffer is selected from the group consisting of 0.5-0.75M Sodium carbonate/bicarbonate

11. A composition of claim 9 wherein Part A comprises a source of calcium ions in the concentration of 20 mM solution in order to activate Factor XIII.

12. The composition of claim 9 wherein said acid buffer is selected from the group consisting of acetic acid.

13. The composition of claim 9 in which divalent ions such as calcium, zinc and magnesium ions markedly increase the rates of fibrin polymerization, the length and strength of fibrin filaments, and fasten scaffold polymerization

14. The composition of claim 9 wherein all components can be sterilized without loosing their properties

15. The composition of claim 9 wherein fibrin monomer concentration is from about 12 mg/ml to 20 mg/ml of acteic acid.

16. The composition of claim 9 wherein their application over tissue is biocompatible

17. The method of claim 3 wherein Part A and Part B when mixed form and adhesive foaming gel.

18. The method of claim 3 wherein the components are cross-linked and the gel is formed in less than 6 seconds and at a gel strength of G′ 2000 dyn/cm2 measured by Theological instruments.

19. The method of claim 3 in which the first and second containers can maintain fibrin sealing components such as Factor XIII, without these compounds loosing their capacity to stabilize a fibrin clot.

20. The composition of claim 9 in which Part A contrains calcium ions that activate Factor XIII

21. A method of 9 in which activated Factor XIII crosslinks the polymerized fibrin monomer.

22. The method of claim 9 wherein the cross-linked matrix formed by Teleostan Fish Gelatin type A, Bovine serum albumin, Carrageenan (sulfonated polysaccharide) Polyvinylpyrrolidinone (PVP), sucrose, Carbomer 934 and ACTIVA in buffer solution serves as scaffold for the fibrin components to bind tissue together without compression or the addition of a suture, a staple, a tape, or a bandage.

23. The method of claim 9 mixture of claim 7 wherein when parts are mixed and activated have three functions; first it spread throughout the cavity in as foam; second, adheres and “sticks” to wet tissue forming a matrix in the pool of blood, and third, promotes the cross linking fibrin in presence of FXIII inducing a fast coagulation and adhesiveness of injured tissue.

24. The method of claim 3 wherein said composition is cured to produce a functional clot within 1 minute from the moment that the gel is applied to the cavity.

25. The method of claim 7, wherein the foam in gel state is induced by a chemical reaction from alginic acid, and or Carbomer 934 and/or carboxylic acid and/or Carboxymethyl cellulose, and Sodium monobasic phosphate (NaH₂PO₄, buffer mixed with a basic solution containing Sodium bicarbonate (NaHCO₃) that releases CO2, in the presence of N-lauroylsarcosine sodium salt and carrageenan type 2 and tween 80 or other ionic surfactants.

26. The method of claim 7 in which the mixing of parts produce a non-exotermic foam that is greater than 400% the volume of the components before the mix.

27. The composition of claim 9, wherein the PVP,sucrose, albumin and gelatin are macromolecules that produce a 3-D polymer which enhances the foam and makes it last longer, while improving tissue adhesivity through matrix formation.

28. The method of claim 7 where the gel strength measured by the rheometric value G′ reaches 2000 dyn/2 cm and maintain the strength for at least 20 minutes.

29. The method of claim 9 wherein the composition containing nondynamic fibrin monomer is substantially free of any exogenous enzyme which catalyzes the formation of fibrin from fibrinogen.

30. A Gas propellant device (kit) for mixing and applying to intracavitary tissues a biocompatible fluid adhesive gelatin foam for therapeutic use, in particular for protecting/cicatrizing tissue wounds and attaching biological tissues to each other, outside the operating room.

31. The method of claim 30 wherein said mixing step is carried out by means of a triple-barrelled syringe, wherein each barrel contains said compositions A,B, and C.

32. A method of claim 30 wherein the mixed composition can be introduced into a cavity to seal lacerated tissue.

33. The method of claim 30 wherein the desired site is selected from the group consisting of skin, abdominal cavity, Torax, cardiovascular system, lymphatic system, pulmonary system, ear, nose, throat, eye, liver, spleen, cranial, spinal, maxillo-facial, bone, tendon, pancreas, genito-urinary tract and alimentary tract.

34. A non-invasive method to seal tissue comprising the following steps to prevent or control blood or other fluid leaks of a) mixing needle implantation; b) mixture of the solutions A+B+C; c) Introduction of the mixture into the cavity, organ or tissue; d) contacting a desired site with a composition which contains nondynamic noncrosslinked fibrin; e) rendering said noncrosslinked fibrin dynamic by neutrilizing its pH which converts noncrosslinked fibrin into fibrin sealant. f) removal of the needle.
